(19) United States
(12) Patent Application Publication
(10) Pub. No.: US 2011/0059097 A1
(43) Pub. Date: Mar. 10, 2011

(54) MFAP4 as a Marker For Regulatory Cells and Anti-Cancer Cells

(75) Inventors: Li Zhang, Toronto (CA); Edward Kim, Toronto (CA); Betty Joe, Toronto (CA)

(73) Assignee: University Health Network

(21) Appl. No.: 12/918,704

(22) PCT Filed: Feb. 20, 2009
(86) PCT No.: PCT/CA09/00197

§ 371 Date: Nov. 22, 2010

Related U.S. Application Data
(60) Provisional application No. 61/030,719, filed on Feb. 22, 2008.

(51) Int. Cl. A61K 39/395; G01N 33/53; A61K 35/12; C07K 16/18; A61P 35/00

(52) U.S. Cl. 424/141.1; 435/7.21; 435/7.24; 424/93.7; 424/93.71; 530/387.1; 530/388.1; 424/130.1

(57) ABSTRACT

The present application provides a novel antibody that is specific for MFAP4. The application also provides methods and uses of MFAP4 as a marker for regulatory cells and/or anti-cancer cells. Further, the application provides methods and uses of MFAP4 binding agents for selection and activation of regulatory cells and/or anti-cancer cells.

### FIELD

The present application relates to a novel antibody, and methods of use thereof. Further, the application relates to methods and uses of MFAP4 as a marker for regulatory cells and anti-cancer cells. The application also relates to MFAP4 binding agents and methods and uses for selecting and activating regulatory cells and anti-cancer cells.

### BACKGROUND

Over the past 20 years, short-term allograft survival has been improved significantly with the advent of immunosuppressive drugs. However, the deterioration of drug efficacy over a prolonged period of time renders them ineffective in preventing chronic graft rejection. Moreover, immunosuppressive drugs render patients susceptible to infections and malignancy. A major goal of transplantation is to induce long-term transplant tolerance without impairing the recipient immune system. Many studies have established the critical role of regulatory T cells in immune tolerance.

The inventors have previously identified and cloned a novel regulatory subset of αβTCR⁺CD4⁻CD8⁻ T (DNT) cells and demonstrated that these cells can kill activated antigen-specific CD4⁺ and CD8⁺ effector T cells in vitro and in vivo (1-5). Adoptive transfer of DNT regulatory cells activated by donor-lymphocyte infusion can potently suppress anti-donor T cell responses in vivo, leading to long-lasting tolerance to transplants (1-5). The inventors have also demonstrated the suppression of autoreactive T cells by DNT regulatory cells in an animal model of autoimmune diabetes, in which peptides activated DNT regulatory cells provided protection against auto-reactive T cells specific for an antigen expressed in pancreatic islets (6). In addition, the inventors have demonstrated that DNT cells have potent anti-cancer effect (7).

DNT cells comprise only ˜1% of PBMC, and no unique surface markers have been identified to positively select DNT cells. Current ex vivo expansion methods for DNT cells rely on depleting multiple subsets from the starting population. Moreover, there are no known functional markers that can distinguish potent suppressive DNT cells within a heterogeneous population during their activation and expansion. The lack of a reliable cell surface marker extends to other regulatory T cell subsets such as CD4⁺CD25⁺Foxp3⁺ T regulatory cells, CD4⁺CD25⁺ T regulatory cells, CD8⁺CD28⁻ T regulatory cells, T regulatory-1 cells, T helper-3 cells, and regulatory NKT cells (reviewed in 8). This makes it difficult to phenotypically distinguish T regulatory cells from other activated effector or memory T cells. For instance, it is still not clear whether Foxp3 is a specific marker for T regulatory cells in humans because activated T cells can transiently express Foxp3 albeit at lower levels than natural CD25^{hi}CD4⁺ T regulatory cells (8). Moreover, Foxp3 is an intracellular molecule whose detection renders cells non-functional for applications subsequent to cell sorting. Other molecules such as CD25, CTLA-4, GITR, and CD127 can be used as cell surface markers for natural CD4⁺CD25⁺ T regulatory cells, however they are unable to fully distinguish natural T regulatory cells from activated T cells because all T cells express these molecules upon activation (8).

Thus, there is a need for methods of detecting and selecting or isolating regulatory cells.

### SUMMARY

The present inventors have identified and developed a novel antibody. Specifically, the inventors have identified and developed a novel antibody that binds MFAP4. Further, the inventors have identified that MFAP4 is expressed on the surface of a subset of regulatory cells and anti-cancer cells. Thus, MFAP4 can be used as a marker to detect, enrich and/or select regulatory cells and/or anti-cancer cells. In addition, the inventors have shown that the regulatory cells and anti-cancer cells can be activated by binding of MFAP4. The activated regulatory cells can be used to treat or prevent the progression of many types of immune-related diseases, such as graft rejection, graft-versus-host disease, autoimmune disease, allergic diseases and infectious disease. The activated anti-cancer cells can be used to treat or prevent the progression of cancer.

The inventors have cloned and sequenced a novel antibody that specifically binds MFAP4 and determined the sequence of the light and heavy chain variable regions and complementarity determining regions 1, 2 and 3 of the antibody.

Accordingly, the application discloses isolated light chain complementarity determining region 1 (CDR1) comprising the amino acid sequence QSLLSSGNQKNY (SEQ ID NO:44); isolated light chain complementarity determining region 2 (CDR2) comprising the amino acid sequence YSS (SEQ ID NO:45); and isolated light chain complementarity determining region 3 (CDR3) comprising the amino acid sequence LQHYSSPFT (SEQ ID NO:46); and isolated heavy chain CDR1 comprising the amino acid sequence GFPFSNYG (SEQ ID NO:21); isolated heavy chain CDR2 comprising the amino acid sequence ISYDGRST (SEQ ID NO:22) and isolated heavy chain CDR3 comprising the amino acid sequence VRHELPEDH (SEQ ID NO:23).

The application also discloses isolated nucleic acid sequences encoding the light chain complementarity determining region 1 (CDR1) comprising the amino acid sequence QSLLSSGNQKNY (SEQ ID NO:44); the light chain complementarity determining region 2 (CDR2) comprising the amino acid sequence YSS (SEQ ID NO:45); and the light chain complementarity determining region 3 (CDR3) comprising the amino acid sequence LQHYSSPFT (SEQ ID NO:46); and the heavy chain CDR1 comprising the amino acid sequence GFPFSNYG (SEQ ID NO:21); the heavy chain CDR2 comprising the amino acid sequence ISYDGRST (SEQ ID NO:22); and the heavy chain CDR3 comprising the amino acid sequence VRHELPEDH (SEQ ID NO:23).

Additional aspects disclosed in the present application are isolated light chain variable regions comprising light chain CDR1, CDR2 and/or CDR3 disclosed herein (SEQ ID NOS:44, 45 and/or 46), and isolated heavy chain variable regions comprising heavy chain CDR1, CDR2 and/or CDR3 disclosed herein (SEQ ID NOS:21, 22 and/or 23). In one embodiment, the light chain variable region comprises the amino acid sequence shown in FIG. 16 (SEQ ID NO:43). In another embodiment, the heavy chain variable region comprises the amino acid sequence shown in FIG. 15 (SEQ ID NO:20).

Another aspect of the present application is a novel binding agent that specifically binds MFAP4, that comprises at least one light chain complementarity determining region disclosed herein (i.e. one or more of SEQ ID NOs:44-46) and/or at least one heavy chain complementarity determining region disclosed herein (i.e. one or more of SEQ ID NOs:21-23). In one embodiment, the binding agent comprises the light chain variable region of SEQ ID NO:43 and the heavy chain variable region of SEQ ID NO:20. In yet a further embodiment, the binding agent is an antibody. In another embodiment, the binding agent is a monoclonal antibody. In yet a further embodiment, the monoclonal antibody is a monoclonal antibody termed G1.

Further, the application provides a method of detecting regulatory and/or anti-cancer cells comprising the steps of:
(1) contacting a test sample with a binding agent that binds specifically to MFAP4 on the cell to produce a binding agent-MFAP4 complex;
(2) detecting the amount of binding agent-MFAP4 complex in the test sample; and
(3) comparing the amount of binding agent-MFAP4 complex in the test sample to a control.

In addition, the application provides a method of selecting or enriching regulatory and/or anti-cancer cells comprising the steps:
(1) contacting a test sample with a binding agent that binds specifically to MFAP4 on the cell to produce a binding agent-MFAP4 complex; and
(2) selecting or enriching regulatory and/or anti-cancer cells by selecting the binding agent-MFAP4 complex from the test sample.

The application also provides a method of activating regulatory and/or anti-cancer cells comprising:
contacting regulatory and/or anti-cancer cells with a binding agent that binds specifically to MFAP4 on the cell to crosslink MFAP4.

In one embodiment, the cells are contacted in vitro.

In one specific embodiment, the novel binding agent disclosed herein is the binding agent for the methods disclosed herein.

In another aspect, the application provides use of an MFAP4-selected or -activated regulatory cell obtained by the method of the application for treating or preventing an immune-related disease. The application also provides a method of treating or preventing an immune-related disease comprising administering an MFAP4-selected or -activated regulatory cell obtained by the method of the application to an animal in need thereof. The application further provides use of an MFAP4-selected or -activated regulatory cell in the preparation of a medicament for treating or preventing an immune-related disease. The application also provides an MFAP4-selected or -activated regulatory cell obtained by the method of the application for use in the treatment or prevention of an immune-related disease.

In yet another aspect, the application provides use of an MFAP4-selected or -activated anti-cancer cell obtained by the method of the application for treating or preventing a cancer. The application also provides a method of treating or preventing a cancer comprising administering an MFAP4-selected or -activated anti-cancer cell obtained by the method of the application to an animal in need thereof. The application further provides use of an MFAP4-selected or -activated anti-cancer cell in the preparation of a medicament for treating or preventing a cancer. The application also provides an MFAP4-selected or -activated anti-cancer cell obtained by the method of the application for use in the treatment or prevention of a cancer.

In a further aspect, the application provides a method of modulating an immune response in an animal comprising administering an effective amount of an MFAP4 binding agent to an animal in need thereof. The application also provides a use of an effective amount of an MFAP4 binding agent for modulating an immune response in an animal in need thereof. The application further provides a use of an effective amount of an MFAP4 binding agent in the preparation of a medicament for modulating an immune response. The application yet further provides an MFAP4 binding agent for use in modulation of an immune response.

In another aspect, the present application provides a method of modulating cancer in an animal comprising administering an effective amount of an MFAP4 binding agent to an animal in need thereof. The application also provides a use of an effective amount of an MFAP4 binding agent for modulating cancer in an animal in need thereof. The application further provides a use of an effective amount of an MFAP4 binding agent in the preparation of a medicament for modulating cancer. The application yet further provides an MFAP4 binding agent for use in modulation of cancer.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows that DNT cell clones but not their mutants can kill A20 B cell lymphoma in vitro and protect against lethal dose of A20 after adoptive transfer in vivo. (A) DNT cell clone CN04 and its natural mutant clone CN48 were used as effector cells at indicated ratios. A20 tumour cells were used as targets in a standard ⁵¹Cr release assay. (B) Mice were injected with a lethal dose of A20 tumor cells either alone or together with 5×10⁵ DNT cell clones or their mutants as indicated. Percent tumor-free survival in each group is shown. Each group contains 5-12 mice.

FIG. 2 shows that G1 mAb selectively stains tumor-reactive and regulatory DNT cell clones, but not their non-functional mutants. (A) DNT cell clone TN12 (unfilled red line, labeled TN12) and its natural mutant TN12.8 (filled black histogram, labeled TN12.8), which has lost cytotoxicity against tumor cells and allo-reactive T cells, were immunostained with G1 mAb and analyzed by flow cytotometry. (B) Immunostaining of another functional DNT cell clone, CN04 (filled histogram labeled CN04), and its natural mutant CN4.8 (filled histogram labeled CN4.8), which has lost cytotoxicity against tumor cells and allo-reactive T cells, with G1 mAb.

FIG. 3 shows ex vivo expansion of DNT cells from PBMC of healthy donors and their anti-tumor function in a xenograft tumor model. (A) Number of DNT cells after 10 days of ex vivo expansion from 5-10 ml of whole blood (n=10). (B) Ex vivo-expanded human DNT cells can inhibit human lung cancer progression in NOD-SCID mice. NOD-SCID mice were subcutaneously injected with 10⁶H460 tumor cells either alone or together with 5×10⁶ ex vivo-expanded CD8⁺ or DNT cells. Percent tumor free survival of the recipients is shown. P=0.0436 between DNT versus CD8 T cell-treated groups.

FIG. 4 shows that G1 mAb selectively immunostains a subset of ex vivo-expanded human DNT cells. Ten days after expansion ex vivo, human DNT cells were immunostained with G1 mAb and analyzed by flow cytometry.

FIG. 5 shows the validation of MFAP4 as the molecule recognized by the functional marker-specific G1 mAb. MFAP4 was identified by sequencing the protein band derived from immunoprecipitating G1⁺ DNT cell clone lysates with G1 mAb. The protein recognized by G1 mAb and anti-MFAP4 mAb are of the same molecular sizes as determined by western blot. Recombinant mouse and human MFAP4 proteins were expressed in CHO cells and whole cell lysates were prepared as positive controls for Western blot analysis using anti-MFAP4 mAb. MFAP4 is expressed by functional G1⁺ DNT cell clones but not G1⁻ DNT cell mutant clones that have lost cytotoxicity against tumor cells and allo-reactive T cells.

FIG. 6 shows that the molecule recognized by the functional marker-specific G1 mAb is specifically detected on the same activated DNT cell that expresses MFAP4, recognized by anti-MFAP4 mAb. DNT cells were purified from B6 mice, activated in vitro for 5 days, and subsequently co-immunostained with G1 mAb and anti-MFAP4 mAb and analyzed by flow cytometry.

FIG. 7 shows that MFAP4 is expressed by a subset of activated DNT cells. Lymph node and spleen cells from B6.lpr mice (in which the majority of CD4 and CD8 T cells were depleted ex vivo) were activated in vitro for 5 days using alloantigen stimulation (irradiated [B6×Balb/c]F1 splenocytes), subsequently immunostained with anti-MFAP4, anti-TCRβ, anti-CD4, anti-CD8, and anti-NK1.1 antibodies, and then analyzed by flow cytometry. Dot plot (top) is gated on βTCR⁺ cells; top highlighted box in the dot plot corresponds to CD4⁺, CD8⁺, NK1.1⁺ (βTCR⁺ cells, within which the smaller top box corresponds to MFAP4⁺ cells; bottom highlighted box corresponds to CD4⁻, CD8⁻, NK1.1⁻ TCRβ⁺ cells (DNT cells), within which the smaller bottom box corresponds to MFAP4⁺ cells. Histogram plot (bottom) is gated on βTCR⁺ cells and shows MFAP4 expression on CD4/CD8/NK1.1⁺ cells (filled light gray histogram), DNT cells (bold black line), and unstained control (dark gray histogram).

FIG. 8 shows MFAP4 is expressed by a subset of regulatory DNT cells converted from allo-activated CD4⁺ T cells. Purified CD4⁺ T cells from B6.Thy1.1 mice were labeled with the proliferation-tracking dye CFSE and activated in vitro for 6 days by alloantigen stimulation (mature bone marrow-derived dendritic cells from [B6×Balb/c]_{F1} mice), subsequently immunostained with anti-MFAP4, anti-βTCR, anti-CD4, anti-CD8, and anti-NK1.1, and then analyzed by flow cytometry. Contour plot (left) is gated on αβTCR⁺ cells; highlighted box within the contour plot corresponds to CD4⁻, CD8⁻, NK1.1⁻, αβTCR⁺ cells (DNT cells). Histogram plot (right) shows MFAP4 expression gated on activated DNT cells based on the contour plot (left).

FIG. 9 shows that cross-linking MFAP4 on the cell surface of activated DNT cells using G1 mAb or anti-MFAP4 mAb induces rapid expression of IFNγ. DNT cells were purified and activated from B6 mice and subsequently seeded in flat-bottom wells coated with the indicated antibodies (10 μg/ml) to induce cross-linking of each molecule, and in the presence of a golgi inhibitor to permit intracellular measurement of IFNγ. After 5 hours of cross-linking, cells were harvested and analyzed for intracellular levels of IFNγ by flow cytometry. G1 mAb treatment induces rapid IFNγ expression in activated DNT cells.

FIG. 10 shows that plate-bound G1 mAb treatment of regulatory DNT cell clones induces rapid adherence and morphological differentiation. DNT regulatory clones were seeded on flat-bottom wells coated with the indicated antibodies (10 μg/ml) for 5 hours and subsequently analyzed by microscopy.

FIG. 11 shows that MFAP4⁺ DNT cells are potent suppressors of syngeneic allo-reactive CD4 T cells. CFSE-labeled B6.Thy1.1⁺ CD4 T cells were used as responders to alloantigen stimulation (irradiated [B6×Balb/c]_{F1} splenocytes) for 5 days, and their proliferation was analyzed by flow cytometry. Indicated doses of alloantigen-activated DNT cells (purified from B6.lpr mice and activated by irradiated [B6×Balb/c]_{F1} splenocytes for 5 days prior) were added as suppressor cells. Bottom two rows (highlighted black box): Activated DNT cells from B6 mice were sorted based on MFAP4 expression into MFAP4⁻ and MFAP4⁺ cells and used respectively as suppressors against syngeneic allo-reactive CD4 T cells. CD4⁺ T cells in the far left column of dot plots are highlighted by a black box (upper right quadrant), and the next two columns (middle and right) correspond to CFSE (cell division tracking dye) fluorescence gated on CD4⁺ T cells.

FIG. 12 shows that enrichment of MFAP4⁺ DNT cells using G1 mAb selects for potent anti-tumor cells. (A) DNT cells were activated in vitro for 6 days by alloantigen stimulation (irradiated [B6×BAlb/c]_{F1} splenocytes) and immunostained with anti-βTCR, anti-CD4, anti-CD8, anti-NK1.1, anti-MFAP4, and G1 mAb and analyzed by flow cytometry. Left contour plot: mAb isotype controls; right contour plot: anti-MFAP4 and G1 mAb marks a subset of DNT cells. G1⁺ and G1⁻ DNT cell populations were sorted and used respectively as effectors against A20 B cell lymphoma cells in a standard ⁵¹Cr-release cytotoxicity assay at the indicated effector:target ratios (B).

FIG. 13 shows the human MFAP4 sequences.

FIG. 14 shows the murine MFAP4 sequences.

FIG. 15 shows the V_{H} alignment and consensus sequences, variable regions and CDRs.

FIG. 16 shows the V_{L} alignment and consensus sequences, variable regions and CDRs.

### DETAILED DESCRIPTION OF THE DISCLOSURE

### (A) Definitions

The term “a cell” includes a single cell as well as a plurality or population of cells.

The term “amino acid” includes all of the naturally occurring amino acids as well as modified amino acids.

The term “antibody” as used herein is intended to include, without limitation, monoclonal antibodies, polyclonal antibodies, chimeric and humanized antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. In one embodiment, the monoclonal antibody is a humanized monoclonal antibody.

The term “humanized antibody” as used herein means that the antibody or fragment comprises human conserved framework regions (alternatively referred to as constant regions) and the hypervariable regions (alternatively referred to as the antigen binding domain) are of non-human origin. For example, the hypervariable region may be from a mouse, rat or other species. The humanization of antibodies from non-human species has been well described in the literature. See for example EP-B1 0 239400 and Carter & Merchant 1997 (Curr Opin Biotechnol 8, 449-454, 1997 incorporated by reference in their entirety herein). Humanized antibodies are also readily obtained commercially (eg. Scotgen Limited, 2 Holly Road, Twickenham, Middlesex, Great Britain.)

Humanized forms of rodent antibodies are readily generated by CDR grafting (Riechmann et al. Nature, 332:323-327, 1988). In this approach the six CDR loops comprising the antigen binding site of the rodent monoclonal antibody are linked to corresponding human framework regions. CDR grafting often yields antibodies with reduced affinity as the amino acids of the framework regions may influence antigen recognition (Foote & Winter. J Mol Biol, 224: 487-499, 1992). To maintain the affinity of the antibody, it is often necessary to replace certain framework residues by site directed mutagenesis or other recombinant techniques and may be aided by computer modeling of the antigen binding site (Co et al. J Immunol, 152: 2968-2976, 1994).

Humanized forms of antibodies are optionally obtained by resurfacing (Pedersen et al. J Mol Biol, 235: 959-973, 1994). In this approach only the surface residues of a rodent antibody are humanized.

The term “anti-cancer cell” as used herein refers to a cell that can kill cancer cells. In one embodiment, the anti-cancer cell is cytotoxic against cancer cells. In a specific embodiment, the anti-cancer cell is a T cell. In a more specific embodiment, the T cell is a double negative (DNT) T cell. The term “DNT cells” include cells that are CD3⁺TCR⁺CD4⁻CD8⁻. In one embodiment, DNT cells are CD3⁺αβTCR⁺CD4⁻CD8⁻. In a specific embodiment, DNT cells are CD3⁺αβTCR⁺CD4⁻CD8⁻NK1.1⁻.

The term “binding agent” as used herein refers to agents that specifically bind to another substance, such as an MFAP4 protein.

The term “cancer cell” includes cancer or tumor-forming cells, transformed cells or a cell, such as a pre-cancerous cell, that is susceptible to becoming a cancer or tumor-forming cell.

The term “control” as used herein refers to a sample that is known as having a particular trait or not having a particular trait. The term also includes a pre-determined standard.

The phrases “detecting regulatory cells” or “detecting anti-cancer cells” refer to a method or process of determining if a sample has or does not have regulatory cells or anti-cancer cells, and includes determining the quantity and/or type of regulatory cells or anti-cancer cells.

The phrases “enrich and/or select regulatory cells” or “enrich and/or select anti-cancer cells” refer to a method or process of increasing the concentration of regulatory cells or anti-cancer cells, or selecting regulatory cells or anti-cancer cells from a sample.

As used herein, the phrase “effective amount” means an amount effective, at dosages and for periods of time necessary to achieve the desired result. Effective amounts of therapeutic may vary according to factors such as the disease state, age, sex, or weight of the animal. Dosage regime may be adjusted to provide the optimum therapeutic response. For example, several divided doses may be administered daily or the dose may be proportionally reduced as indicated by the exigencies of the therapeutic situation.

The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded, and represents the sense or antisense strand. Further, the term “nucleic acid” includes the complementary nucleic acid sequences.

The term “isolated polypeptides” refers to a polypeptide substantially free of cellular material or culture medium when produced by recombinant DNA techniques, or chemical precursors or other chemicals when chemically synthesized.

The term “label” is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “MFAP4” or “microfibrillar-associated protein-4” refers to a 255-amino acid glycoprotein of the fibrinogen-related domain family of proteins. MFAP4 contains a C-terminal fibrinogen-like domain (which has high similarity to the C-terminal halves of fibrinogen β and γ chains) and the N-terminal Arg-Gly-Asp (RGD) motif sequence that serves as an integrin-binding domain, suggesting that it is an extracellular matrix protein involved in cell adhesion or intercellular interactions. In one embodiment, the MFAP4 is of murine origin. For example, the amino acid sequence of SEQ ID NO:4. In another embodiment, the MFAP4 is of human origin. For example, the amino acid sequence of SEQ ID NO:1. The term also includes variants of SEQ ID NOS:1 and 4. In one embodiment, the variant amino acid sequence has at least 70%, preferably at least 80%, more preferably at least 85%, even more preferably at least 90% and even most preferably at least 95% sequence identity to SEQ ID NO:1 or 4.

The term “heavy chain variable region” or “V_{H} region” as used herein refers to the variable region of a heavy chain of an antibody molecule. The heavy chain variable region has three complementarity determining regions (CDRs). The term “heavy chain complementarity determining region” as used herein refers to regions of hypervariability within the heavy chain variable region of an antibody molecule. The heavy chain variable region has three complementarity determining regions termed heavy chain complementarity determining region 1, heavy chain complementarity determining region 2 and heavy chain complementarity determining region 3 from the amino terminus to carboxy terminus.

The term “light chain variable region” or “V_{L} region” as used herein refers to the variable region of a light chain of an antibody molecule. Light chain variable regions have three complementarity determining regions. The term “light chain complementarity determining region” as used herein refers to regions of hypervariability within the light chain variable region of an antibody molecule. Light chain variable regions have three complementarity determining regions termed light chain complementarity determining region 1, light chain complementarity determining region 2 and light chain complementarity determining region 3 from the amino terminus to the carboxy terminus.

The term “nucleic acid sequence” as used herein refers to a sequence of nucleoside or nucleotide monomers consisting of naturally occurring bases, sugars and inter-sugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The term “regulatory cells” as used herein refers to cells that have immune regulatory function. In one embodiment, the regulatory cells are regulatory T cells, which include, without limitation, CD4⁺CD25⁺ T regulatory cells, CD4⁺CD25⁺Foxp3⁺ T regulatory cells, T regulatory-1 (Tr1) cells, T helper-3 (Th3) cells, CD4⁺CD25⁻ T regulatory cells, CD8⁺CD28⁻ T regulatory cells, CD8αα⁺ T regulatory cells, γδ regulatory T cells, regulatory natural killer (NK) T cells and double negative T (DNT) cells. In a specific embodiment, the regulatory T cells are DNT cells. The term “DNT cells” include cells that are CD3⁺TCR⁺CD4⁻CD8⁻. In one embodiment, DNT cells are CD3⁺αβTCR⁺CD4⁻CD8⁻. In a specific embodiment, DNT cells are CD3⁺αβTCR⁺CD4⁻CD8⁻NK1.1⁻.

The term “sample” or “test sample” as used herein refers to any fluid, cell or tissue sample from a subject that contain regulatory and/or anti-cancer cells. For example, the sample can be blood, serum, plasma, lymphatic fluid, or any tissue or cell type. The term also includes cell lines.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score−50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “subject” as used herein refers to any member of the animal kingdom, preferably a mammal, more preferably a human being.

The term “treating or preventing” includes, but is not limited to, alleviation or amelioration of one or more symptoms or conditions of a disease or condition (such as cancer, graft rejection, autoimmune disease, graft versus host disease, allergy, infection etc.), diminishment of extent of disease, stabilized state of disease, preventing spread of disease, delaying or slowing of disease progression, and amelioration or palliation of the disease state, remission whether detectable or undetectable and/or prolonged survival as compared to expected survival if not receiving treatment.

As used herein, the phrase “treating or preventing immune related diseases” refers to inhibiting the disease, preventing the disease, decreasing the severity of the disease or improving disease symptoms. The term “immune related diseases” as used herein includes, without limitation, graft rejection, graft versus host disease, autoimmune disease (e.g. diabetes, lupus, arthritis, multiple sclerosis, lymphoproliferative diseases, etc.), allergic diseases and infectious diseases. The term “treating or preventing cancer” refers to inhibiting cancer cell replication, preventing transformation of a cell to a cancer-forming cell, inhibiting cancer spread (metastasis), inhibiting tumor growth, reducing cancer cell number or tumor growth, decreasing the malignant grade of a cancer (e.g., increased differentiation), or improving cancer-related symptoms. In one embodiment, the cancer is breast cancer. In another embodiment, the cancer is blood cancer or epithelial cancer. In yet another embodiment, the blood cancer is leukemia or lymphoma. In a further embodiment, the epithelial cancer is melanoma, esophageal, or lung cancer. In yet a further embodiment, the immune related disease involves graft rejection. Thus, the term “treating or preventing graft rejection” refers to inhibiting graft rejection, preventing graft rejection, reducing the severity of graft rejection, or improving graft rejection-related symptoms.

The term “variant” as used herein includes modifications or chemical equivalents of the amino acid and nucleic acid sequences disclosed herein that perform substantially the same function as the polypeptides or nucleic acid molecules disclosed herein in substantially the same way. In one embodiment, variants of polypeptides disclosed herein include, without limitation, conservative amino acid substitutions. Variants of polypeptides also include additions and deletions to the polypeptide sequences disclosed herein. In addition, variant nucleotide sequences and polypeptide sequences include analogs and derivatives thereof.

### (B) Complementarity Determining Regions and Variable Regions and Nucleic Acids Thereof

The application provides isolated light chain complementarity determining region 1 (CDR1) comprising the amino acid sequence QSLLSSGNQKNY (SEQ ID NO:44); isolated light chain complementarity determining region 2 (CDR2) comprising the amino acid sequence YSS (SEQ ID NO:45); and isolated light chain complementarity determining region 3 (CDR3) comprising the amino acid sequence LQHYSSPFT (SEQ ID NO:46); and isolated heavy chain CDR1 comprising the amino acid sequence GFPFSNYG (SEQ ID NO:21); isolated heavy chain CDR2 comprising the amino acid sequence ISYDGRST (SEQ ID NO:22); and isolated heavy chain CDR3 comprising the amino acid sequence VRHELPEDH (SEQ ID NO:23). In one embodiment, the V_{L} region comprises the sequence shown in SEQ ID NO:43. In another embodiment, the V_{H} region comprises the sequence shown in SEQ ID NO:20.

The application provides isolated light chain complementarity determining region 1 (CDR1) consisting of the amino acid sequence QSLLSSGNQKNY (SEQ ID NO:44); isolated light chain complementarity determining region 2 (CDR2) consisting of the amino acid sequence YSS (SEQ ID NO:45); and isolated light chain complementarity determining region 3 (CDR3) consisting of the amino acid sequence LQHYSSPFT (SEQ ID NO:46); and isolated heavy chain CDR1 consisting of the amino acid sequence GFPFSNYG (SEQ ID NO:21); isolated heavy chain CDR2 consisting of the amino acid sequence ISYDGRST (SEQ ID NO:22); and isolated heavy chain CDR3 consisting of the amino acid sequence VRHELPEDH (SEQ ID NO:23). In one embodiment, the V_{L} region consists of the sequence shown in SEQ ID NO:43. In another embodiment, the V_{H} region consists of the sequence shown in SEQ ID NO:20.

The application also discloses variants of the CDR sequences disclosed above. For example, the variants include polypeptides that can bind to the same epitope or antigen recognized by the CDR sequences disclosed above.

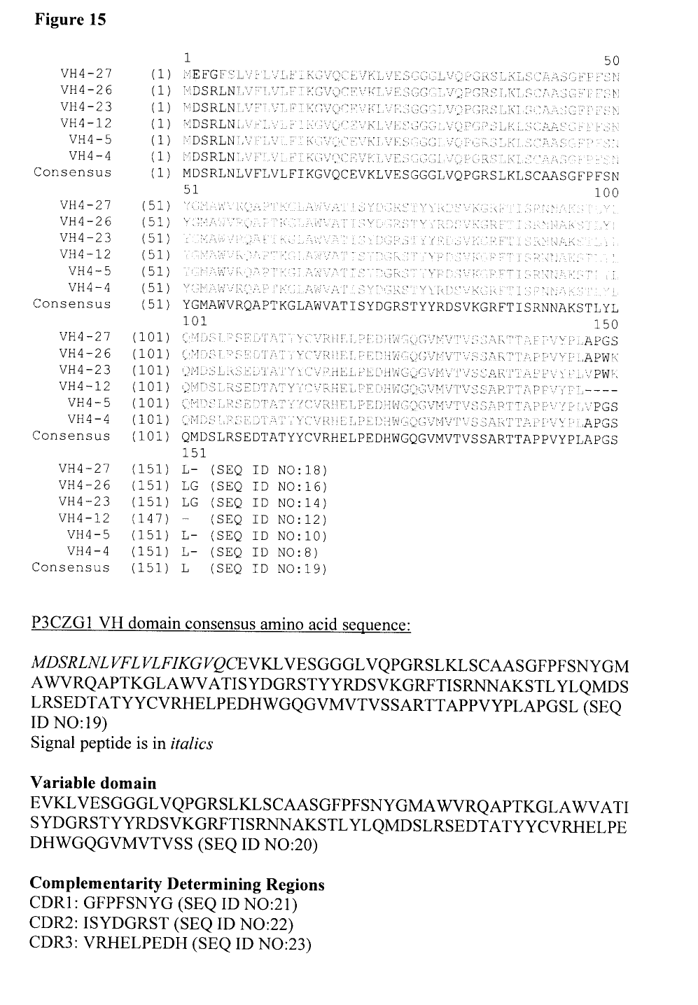

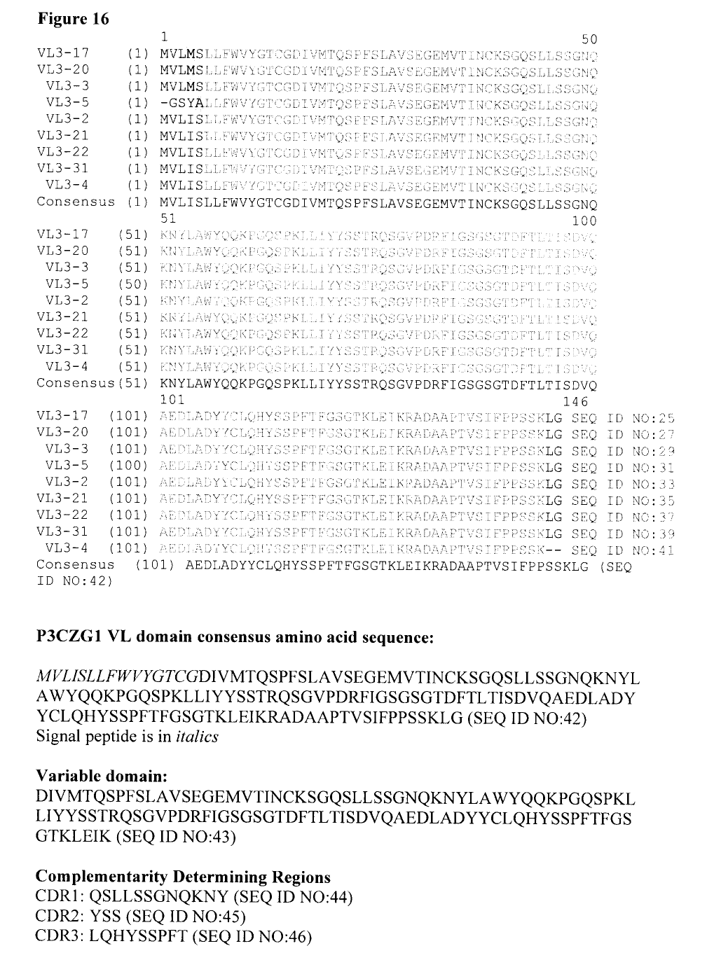

Additional aspects disclosed in the present application are isolated light chain variable regions comprising light chain CDR1, CDR2 and/or CDR3 disclosed herein (SEQ ID NOS:44, 45 and/or 46), and isolated heavy chain variable regions comprising heavy chain CDR1, CDR2 and/or CDR3 disclosed herein (SEQ ID NOS:21, 22 and/or 23). In one embodiment, the light chain variable region comprises the amino acid sequence shown in FIG. 16 (SEQ ID NO:43). In another embodiment, the heavy chain variable region comprises the amino acid sequence shown in FIG. 15 (SEQ ID NO:20). In one embodiment, the light chain variable region consists of the amino acid sequence shown in FIG. 16 (SEQ ID NO:43). In another embodiment, the heavy chain variable region consists of the amino acid sequence shown in FIG. 15 (SEQ ID NO:20).

The application also discloses variants of the isolated light chain variable regions and heavy chain variable regions disclosed above. For example, the variants include polypeptides that can bind to the same epitope or antigen recognized by the isolated light chain variable regions and isolated heavy chain variable regions disclosed above.

In one embodiment, the variant amino acid sequences of the light chain CDR1, CDR2 and CDR3, and the heavy chain CDR1, CDR2 and CDR3 have at least 50%, preferably at least 60%, more preferably at least 70%, most preferably at least 80%, even more preferably at least 90%, and even most preferably 95% sequence identity to SEQ ID NOs:44-46 or 21-23.

In another embodiment, the variant amino acid sequences of the light chain variable region and the heavy chain variable region have at least 50%, preferably at least 60%, more preferably at least 70%, most preferably at least 80%, even more preferably at least 90% and even most preferably 95% sequence identity to SEQ ID NO:20 or 43.

The application also discloses isolated nucleic acid sequences encoding the light chain complementarity determining region 1 (CDR1) comprising the amino acid sequence QSLLSSGNQKNY (SEQ ID NO:44); the light chain complementarity determining region 2 (CDR2) comprising the amino acid sequence YSS (SEQ ID NO:45); and the light chain complementarity determining region 3 (CDR3) comprising the amino acid sequence LQHYSSPFT (SEQ ID NO:46); and the heavy chain CDR1 comprising the amino acid sequence GFPFSNYG (SEQ ID NO:21); the heavy chain CDR2 comprising the amino acid sequence ISYDGRST (SEQ ID NO:22); and the heavy chain CDR3 comprising the amino acid sequence VRHELPEDH (SEQ ID NO:23).

The application also provides isolated nucleic acid sequences encoding variants of the CDR sequences and variable region sequences discussed above.

Variant nucleic acid sequences include nucleic acid sequences that hybridize to the nucleic acid sequences encoding the amino acid sequences of SEQ ID NOS:44-46 or 21-23 under at least moderately stringent hybridization conditions, or have at least 50%, 60%, 70%, 80%, 90% or 95% sequence identity to the nucleic acid sequences that encode the amino acid sequence of SEQ ID NOS:44-46 and 21-23.

The application also discloses an isolated nucleic acid sequence encoding the light chain variable region disclosed herein and an isolated nucleic acid sequence encoding the heavy chain variable region disclosed herein. In one embodiment, the isolated nucleic acid sequence encodes the light chain variable region comprising the amino acid sequence shown in FIG. 16 (SEQ ID NO:43). In another embodiment, isolated nucleic acid sequence encodes the heavy chain variable region comprising the amino acid sequence shown in FIG. 15 (SEQ ID NO:20).

The application also discloses variants of the nucleic acid sequences that encode for the light chain variable region and heavy chain variable region disclosed herein. For example, the variants include nucleotide sequences that hybridize to the nucleic acid sequences encoding the light chain variable region and heavy chain variable region disclosed herein under at least moderately stringent hybridization conditions. In another embodiment, the variant nucleic acid sequences have at least 50%, preferably at least 70%, most preferably at least 80%, even more preferably at least 90% and even most preferably at least 95% sequence identity to the nucleic acid sequence encoding the amino acid sequence of SEQ ID NO: 43 or SEQ ID NO:20.

A person skilled in the art will appreciate that the novel nucleic acid sequences of the present application can be used in a number of recombinant methods.

Accordingly, the nucleic acid sequences of the present application may be incorporated in a known manner into an appropriate expression vector which ensures good expression of the proteins encoded thereof. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the present application and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The present application therefore contemplates a recombinant expression vector of the present application containing a nucleic acid molecule of the present application, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in (Goeddel, 1990), Gene Expression Technology Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the present application may also contain a selectable marker gene which facilitates the selection of host cells transformed or transfected with a recombinant molecule of the present application. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the present application and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector of the present application. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in (Sambrook et al., 2001) (Molecular Cloning: A Laboratory Manual, 3rd Edition, Cold Spring Harbor Laboratory Press, 2001), and other laboratory textbooks.

Suitable host cells include a wide variety of eukaryotic host cells and prokaryotic cells. For example, the proteins of the present application may be expressed in yeast cells or mammalian cells. Other suitable host cells can be found in (Goeddel, 1990), Gene Expression Technology: Methods in Enzymology 185, Academic Press, San Diego, Calif. (1991). In addition, the proteins of the present application may be expressed in prokaryotic cells, such as Escherichia coli (Zhang et al., 2004), Science 303(5656): 371-3). In addition, a Pseudomonas based expression system such as Pseudomonas fluorescens can be used (US Patent Application Publication No. US 2005/0186666, (Schneider et al., 2005)).

Yeast and fungi host cells suitable for carrying out the present application include, but are not limited to Saccharomyces cerevisiae, the genera Pichia or Kluyveromyces and various species of the genus Aspergillus. Examples of vectors for expression in yeast S. cerevisiae include pYepSec1 ((Baldari et al., 1987), Embo J. 6:229-234), pMFa ((Kurjan and Herskowitz, 1982), Cell 30:933-943 (1982)), pJRY88 ((Schultz et al., 1987), Gene 54:113-123), and pYES2 (Invitrogen Corporation, San Diego, Calif.). Protocols for the transformation of yeast and fungi are well known to those of ordinary skill in the art (see (Hinnen et al., 1978) Proc. Natl. Acad. Sci. USA 75:1929); ((Ito et al., 1983), J. Bacteriology 153:163) and ((Cullen et al., 1987) BiolTechnology 5:369).

Mammalian cells suitable for carrying out the present application include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 ((Seed, 1987). Nature 329:840) and pMT2PC ((Kaufman et al., 1987), EMBO J. 6:187-195).

Given the teachings provided herein, promoters, terminators, and methods for introducing expression vectors of an appropriate type into plant, avian, and insect cells may also be readily accomplished. For example, within one embodiment, the proteins of the present application may be expressed from plant cells (see (Sinkar et al., 1987), J. Biosci (Bangalore) 11: 47-58), which reviews the use of Agrobacterium rhizogenes vectors; see also ((Zambryski et al., 1984), Genetic Engineering, Principles and Methods, Hollaender and Setlow (eds.), Vol. VI, pp. 253-278, Plenum Press, New York), which describes the use of expression vectors for plant cells, including, among others, PAPS2022, PAPS2023, and PAPS2034).

Insect cells suitable for carrying out the present application include cells and cell lines from Bombyx, Trichoplusia or Spodotera species. Baculovirus vectors available for expression of proteins in cultured insect cells (SF 9 cells) include the pAc series ((Smith et al., 1983), Mol. Cell. Biol. 3:2156-2165) and the pVL series ((Luckow and Summers, 1989), Virology 170:31-39). Some baculovirus-insect cell expression systems suitable for expression of the recombinant proteins of the present application are described in PCT/US/02442.

Alternatively, the proteins of the present application may also be expressed in non-human transgenic animals such as rats, rabbits, sheep and pigs ((Hammer et al., 1985). Nature 315:680-683); (Brinster et al., 1985; Palmiter and Brinster, 1985; Palmiter et al., 1983) Science 222: 809-814); and ((Leder and Stewart, 1988) U.S. Pat. No. 4,736,866).

Accordingly, the present application provides a recombinant expression vector comprising one or more of the novel nucleic acid sequences disclosed herein as well as methods and uses of the expression vectors in the preparation of recombinant proteins. Further, the application provides a host cell comprising one or more of the novel nucleic acid sequences or expression vectors comprising one or more of the novel nucleic acid sequences.

### (C) Novel Binding Proteins for MFAP4

As mentioned above, the present inventors have identified a novel antibody that binds MFAP4. Further, the inventors have identified that MFAP4 is expressed on the surface of a subset of regulatory cells and/or anti-cancer cells. Thus, MFAP4 can be used as a marker to detect, enrich and/or select regulatory cells and/or anti-cancer cells. In addition, the inventors have shown that the regulatory cells and anti-cancer cells can be activated by binding of MFAP4. The activated regulatory cells can be used to treat or prevent the progression of many types of immune related diseases, such as cancer, graft rejection, graft versus host disease, autoimmune disease, allergic diseases and infectious diseases. The activated anti-cancer cells can be used to treat or prevent cancer.

Accordingly, the application provides a binding agent that specifically binds MFAP4. In one embodiment, the binding agent binds MFAP4 protein having the amino acid sequence of SEQ ID NO:1 or 4. In another embodiment, the binding agent comprises at least one light chain complementarity determining region disclosed herein (i.e. one or more of SEQ ID NOs:44-46) and/or at least one heavy chain complementarity determining region disclosed herein (i.e. one or more of SEQ ID NOs:21-23).

In one embodiment, the binding protein comprises the light chain CDR sequences of SEQ ID NOS:44, 45 and 46 and/or the heavy chain CDR sequences of SEQ ID NOS:21, 22 and 23. In another embodiment, the binding protein comprises the amino acid of SEQ ID NO: 43 (light chain variable region) and/or the amino acid of SEQ ID NO:20 (heavy chain variable region).

A person skilled in the art will appreciate that the application includes variants to the specific binding proteins disclosed above, including chemical equivalents to the sequences disclosed above that perform substantially the same function as the binding proteins disclosed above in substantially the same way. For example, the functional variant of a binding protein will be able to bind to the same antigens or epitopes as the binding proteins disclosed above.

In another embodiment, the binding agent is an antibody or fragment thereof. In yet another embodiment, the binding agent is a monoclonal antibody. In yet a further embodiment, the binding agent is the novel monoclonal antibody, termed G1. In yet another embodiment, the antibody is a humanized monoclonal antibody.

A person skilled in the art will appreciate that other antibodies can be generated that bind to MFAP4. For example, to produce human monoclonal antibodies, antibody producing cells (lymphocytes) can be harvested from a human having cancer and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with cancer cells and the monoclonal antibodies can be isolated. Further, specific antibodies, or antibody fragments, reactive against particular antigens or molecules, such as MFAP4, may also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)).

The present application also includes the use of the novel nucleic acid sequences for the preparation of binding proteins and methods thereof.

### (D) Methods of Selection, Enrichment and Activation

The application provides a method of detecting regulatory and/or anti-cancer cells comprising the steps of:
(1) contacting a test sample with a binding agent that binds specifically to MFAP4 on the cell to produce a binding agent-MFAP4 complex;
(2) detecting the amount of binding agent-MFAP4 complex in the test sample; and
(3) comparing the amount of binding agent-MFAP4 complex in the test sample to a control.

The term “detecting the amount” as used herein includes both qualitative and quantitative measurements. For example, the term includes both detecting whether the complex is present and the quantity of complex present.

The complex can be measured or detected directly or indirectly. For example, the binding agent can be labeled. In another embodiment, a secondary detection agent (e.g. a secondary antibody that is specific for the binding agent) is used and contains a detectable label that can be used to detect the complex.

A person skilled in the art will appreciate that the control can be a sample known to include or not to include regulatory cells or anti-cancer cells. In addition, the control can include binding agents that are known to or not to bind MFAP4.

A further aspect of the application is a method of selecting or enriching regulatory cells and/or anti-cancer cells comprising the steps:
(1) contacting a test sample with a binding agent that binds specifically to MFAP4 on the cell to produce a binding agent-MFAP4 complex; and
(2) selecting or enriching regulatory cells and/or anti-cancer cells by selecting the binding agent-MFAP4 complex from the test sample.

A person skilled in the art will appreciate that the regulatory cells and/or anti-cancer cells are indirectly selected by binding of the binding agent to MFAP4 on the surface of the regulatory cells and/or anti-cancer cells. In one embodiment, the binding agent is labeled. In a further embodiment, the binding agent is immobilized on a solid surface. A number of methods can be used to select or enrich the regulatory and/or anti-cancer cells from the test sample, including, without limitation flow cytometry, immunoprecipitation, magnetic sorting and/or panning.

The application also provides a method of activating regulatory and/or anti-cancer cells comprising:
contacting regulatory and/or anti-cancer cells with a binding agent that binds specifically to MFAP4 on the cell to crosslink MFAP4.

In one embodiment, the cells are contacted in vitro. In another embodiment, the binding agent is administered to a subject and the cells are contacted in vivo.

A person skilled in the art will appreciate that the activation of the regulatory cells and/or anti-cancer cells can be assessed. For example, activated regulatory cells and activated anti-cancer cells have increased expression of IFNγ, changes in morphological/cytoskeletal appearance, such as increased adherence and cell-spreading, and/or increased regulatory activity (e.g. ability to suppress allo-reactive T cells) and/or increased anti-cancer activity (e.g. ability to kill cancer cells).

In one embodiment, the binding agent is immobilized on a solid support to induce crosslinking of MFAP4. A person skilled in the art will appreciate that other methods can be used to induce crosslinking of MFAP4, and hence activation of the regulatory cells and/or anti-cancer cells. For example, a secondary antibody specific for the binding agent can be used to induce crosslinking.

### (E) Uses of MFAP4-Selected and/or -Activated Cells and/or MFAP4 Binding Agents

The MFAP4-selected and/or -activated regulatory cells derived from the methods disclosed herein can be used to treat or prevent immune-related diseases. The MFAP4-selected and/or -activated anti-cancer cells can be used to treat or prevent cancer. For example, see U.S. Pat. No. 6,953,576 and WO 2007/056854, incorporated herein by reference.

Accordingly, in an embodiment, the application provides use of an effective amount of an MFAP4-selected and/or -activated regulatory cell obtained by the method of the application for treating or preventing an immune-related disease. The application also provides a method of treating or preventing an immune-related disease comprising administering an effective amount of an MFAP4-selected and/or -activated regulatory cell obtained by the method of the application for treating or preventing an immune-related disease. The application further provides use of an MFAP4-selected and/or -activated regulatory cell obtained according to the method of the application in the preparation of a medicament for treating or preventing an immune-related disease. The application even further provides an MFAP4-selected and/or -activated regulatory cell obtained by the method of the application for use in the treatment or prevention of an immune-related disease.

The application also provides use of an effective amount of an MFAP4-selected and/or -activated anti-cancer cell obtained by the method of the application for treating or preventing cancer. The application also provides a method of treating or preventing cancer comprising administering an effective amount of an MFAP4-selected and/or -activated anti-cancer cell obtained by the method of the application for treating or preventing cancer. The application further provides use of an MFAP4-selected and/or -activated anti-cancer cell obtained according to the method of the application in the preparation of a medicament for treating or preventing cancer. The application even further provides an MFAP4-selected and/or -activated anti-cancer cell obtained by the method of the application for use in the treatment or prevention of cancer.

In the treatment or prevention of cancer, the cancer that can be treated can be any cancer that is amenable to treatment with DNT cells either alone or in combination with other treatment such as surgery, radiation therapy or chemotherapy, etc. Examples of cancers that may be treated or prevented according to the present application include, but are not limited to, leukemias including chronic myelogenous leukemia, acute myelogenous leukemia, acute lymphoblastic leukemia, and T cell and B cell leukemias, lymphomas (Hodgkins and non-Hodgkins), lymphoproliferative disorders, plasmacytomas, histiocytomas, melanomas, adenomas, sarcomas, carcinomas of solid tissues, hypoxic tumours, squamous cell carcinomas, genitourinary cancers such as cervical, ovarian, and bladder cancers, breast and lung cancers, colon cancer, esophageal cancer, hematopoietic cancers, head and neck cancers, and nervous system cancers.

The application also includes other therapeutic uses of the MFAP4-selected and/or -activated regulatory cells disclosed herein such as the treatment of infectious diseases and for modulating an immune response for example in the treatment of autoimmune diseases, allergies, graft rejection and graft-versus-host disease. In such cases, the method of preparing the MFAP4-selected and/or -activated cells can include adding the appropriate infectious agent, allergen cells or tissues as the antigen.

Accordingly, in one embodiment, the present application provides a method of treating or preventing an infectious disease comprising administering an effective amount of an MFAP4-selected and/or -activated regulatory cell obtained by the method of the present application to an animal in need thereof. The present application also includes a use of an effective amount of an MFAP4-selected and/or -activated regulatory cell obtained by the method of the present application for treating or preventing an infectious disease. The present application further includes a use of an effective amount of an MFAP4-selected and/or -activated regulatory cell obtained by the method of the present application in the manufacture of a medicament for treating or preventing an infectious disease. The present application yet further includes an MFAP4-selected and/or -activated regulatory cell for use in the treatment or prevention of an infectious disease.

In a further embodiment, the present application provides a method of modulating an immune response comprising administering an effective amount of an MFAP4-selected and/or -activated regulatory cell obtained by the method of the present application to an animal in need thereof. The present application also includes a use of an effective amount of an MFAP4-selected and/or -activated regulatory cell obtained by the method of the present application for modulating an immune response. The present application further includes a use of an effective amount of an MFAP4-selected and/or -activated regulatory cell obtained by the method of the present application in the manufacture of a medicament for modulating an immune response. The present application yet further includes an MFAP4-selected and/or -activated regulatory cell for use in modulation of an immune response.

In one embodiment, the MFAP4-selected and/or -activated cells are used to treat an autoimmune disease. Autoimmune diseases that may be treated according to the present application include, but are not limited to, diabetes, arthritis, multiple sclerosis, lupus erythematosus, inflammatory bowel disease, dermatitis, meningitis, thrombotic thrombocytopenic purpura, Sjögren's syndrome, encephalitis, uveitis, leukocyte adhesion deficiency, rheumatic fever, Reiter's syndrome, progressive systemic sclerosis, primary biliary cirrhosis, necrotizing vasculitis, myasthenia gravis, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Raynaud's syndrome, glomerulonephritis, dermatomyositis, chronic active hepatitis, celiac disease, tissue specific autoimmunity, degenerative autoimmunity delayed hypersensitivities, autoimmune complications of AIDS, atrophic gastritis, ankylosing spondylitis and Addison's disease.

In another embodiment, the MFAP4-selected and/or -activated regulatory cells can be used to treat graft-versus-host disease wherein the immune cells in the transplant mount an immune attack on the recipient's immune system. This can occur when the tissue to be transplanted contains immune cells such as when bone marrow or lymphoid tissue is transplanted when treating leukemias, aplastic anemias and enzyme or immune deficiencies, for example.

In a further embodiment, the MFAP4-selected and/or -activated regulatory cells can be used to treat an allergic reaction. In an allergic reaction, the immune system mounts an attack against a generally harmless, innocuous antigen or allergen. Allergies that may be prevented or treated using the methods of the application include, but are not limited to, hay fever, asthma, atopic eczema as well as allergies to poison oak and ivy, house dust mites, bee pollen, nuts, shellfish, penicillin and numerous others.

The MFAP4 binding agents described herein could also be used to modulate regulatory and/or anti-cancer cells in vivo. Accordingly, in another aspect, the present application provides a method of modulating an immune response in an animal comprising administering an effective amount of an MFAP4 binding agent to an animal in need thereof. The application also provides a use of an effective amount of an MFAP4 binding agent for modulating an immune response in an animal in need thereof. The application further provides a use of an effective amount of an MFAP4 binding agent in the preparation of a medicament for modulating an immune response. The application yet further provides an MFAP4 binding agent for use in modulation of an immune response.

In a further aspect, the present application provides a method of modulating cancer in an animal comprising administering an effective amount of an MFAP4 binding agent to an animal in need thereof. The application also provides a use of an effective amount of an MFAP4 binding agent for modulating cancer in an animal in need thereof. The application further provides a use of an effective amount of an MFAP4 binding agent in the preparation of a medicament for modulating cancer. The application yet further provides an MFAP4 binding agent for use in modulation of cancer.

For example, the MFAP4 binding agents may activate or target regulatory cells and/or anti-cancer cells when administered to an area of interest, such as lymphoid tissues or non-lymphoid tissues for regulating allo- or auto-reactive T cell responses, or in tumors for activating tumor-reactive cells.

The term “modulating an immune response” as used herein refers to activating or inhibiting immune cell activity and includes, without limitation, modulating an immune response in the treatment of an immune-related disease.

In one embodiment, modulating an immune response comprises activating an immune response. In another embodiment, modulating an immune response comprises inhibiting an immune response.

The term “modulating a cancer” as used herein refers to activating or inhibiting cancer cells.

The MFAP4-selected or -activated regulatory and/or anti-cancer cells and/or MFAP4 binding agents described herein may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. The compositions may be administered in a convenient manner preferably by injection such as intravenous, subcutaneous, intramuscular, etc.

The compositions described herein can be prepared by per se known methods for the preparation of pharmaceutically acceptable compositions which can be administered to subjects, such that an effective quantity of the cells is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

An effective amount of the composition may vary according to factors such as the disease state, age, sex, and weight of the individual, and the ability of the cells to elicit a desired response in the individual. Dosage regime may be adjusted to provide the optimum therapeutic response. For example, several divided doses may be administered daily or the dose may be proportionally reduced as indicated by the exigencies of the therapeutic situation.

The pharmaceutical compositions of the application may include other active agents that are useful in treating the disease or condition to be treated. For example, in the treatment of a cancer, other anti-cancer agents may be administered either in the same composition or in a separate composition.

In one specific embodiment, the novel antibody, G1, disclosed herein is the binding agent for the methods disclosed herein. The G1 monoclonal antibody comprises the light chain variable region of SEQ ID NO:43 and the heavy chain variable region of SEQ ID NO:20.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

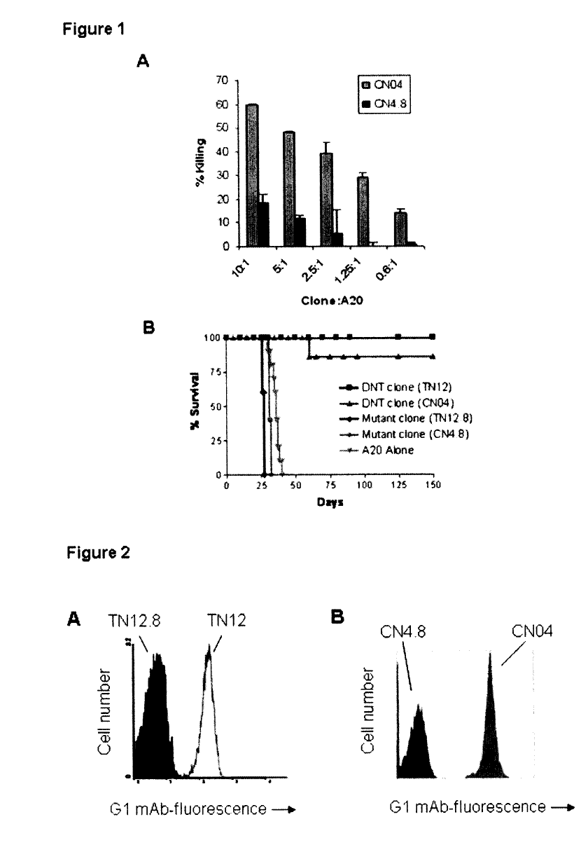

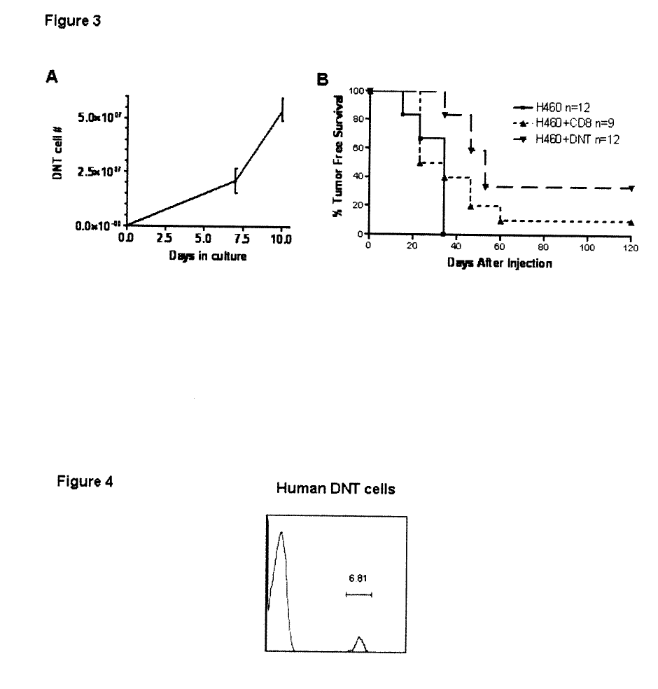

DNT cells share similarity to CD4⁺CD25⁺ T regulatory cells in their potent immune regulatory function. In tumor immunity, however, DNT cells appear to play a different role from that of CD4⁺CD25⁺ T cells. The present inventors have demonstrated that DNT cells in mice possess potent cytotoxicity against syngeneic and allogeneic tumor cell lines in vitro, and that a single infusion of DNT cells can protect recipient mice from a lethal dose of a B cell lymphoma cell line in vivo (FIG. 1; ref. 7). In addition, the present inventors have developed a protocol by which human DNT cells can be purified from peripheral blood and expanded ex vivo (FIG. 3A). Such ex vivo expanded DNT cells are cytotoxic against human leukemia, lymphoma, melanoma, esophagus and lung cancer cell lines, and adoptive transfer of these cells can significantly improve survival and inhibit cancer progression in a xeno-SCID mouse model (FIG. 3B). These data provide compelling evidence of the unique potential for utilizing DNT cells in anti-tumor adoptive cellular immunotherapy. Indeed, new immune cell-mediated therapy may be an effective alternative means of improving survival by eliminating residual cancer cells after chemotherapy-induced remission.

As mentioned above, DNT cells comprise only ˜1% of PBMC, and no unique surface markers have been identified to positively select DNT cells. Thus, the identification of functional markers that can distinguish conventional activated T cells from activated regulatory T cells would facilitate positive selection and enrichment of ex vivo-expanded DNT regulatory cells with potent efficacy and thereby, as one example, potentially increase the in vivo efficacy of DNT cell adoptive therapy.

To facilitate identification of functional markers, a panel of regulatory DNT cell clones has been generated (7). During long-term culture, several natural DNT mutant clones arose that have lost suppressive function against antigen-specific T cells (7). While a single infusion of DNT cell clones prevented the rejection of skin and cardiac allografts in a donor-specific manner, adoptive transfer of the same number of mutant clones had no protective effect (1, 7, 9). Moreover, a single infusion of DNT cell clones prevented the development of B cell lymphoma from a lethal injection of B cell tumor cells, adoptive transfer of the same number of mutant clones had no protective effect (14; FIG. 1). DNT cell clones and their spontaneously-derived mutants therefore provided an excellent system for the identification of specific markers that are important for DNT cell function. A large panel of monoclonal antibodies (mAbs) was thus generated against cell surface molecules on tumor-reactive DNT cells. One mAb (clone: G1) was selected for further characterization, which exhibits strong selective cell surface expression on tumor-reactive and regulatory DNT cell clones, as determined by flow cytometry (FIG. 2).

### 1. Identification and Validation of MFAP4 as Novel Functional Cell Marker Recognized by G1 mAb (FIGS. 4-8)

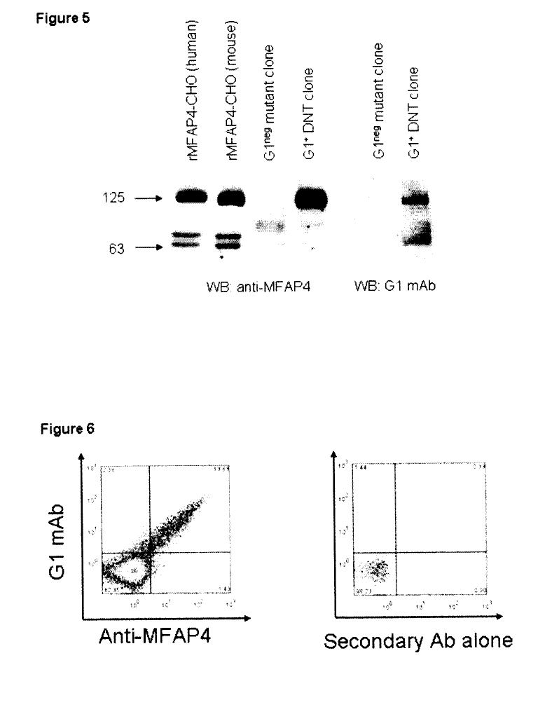

To determine the identity of the DNT cell marker recognized by G1 mAb, membrane fractions of cell lysates from both DNT and mutant cell clones were immunoprecipitated with G1 mAb, resolved by 1-dimensional SDS-PAGE, and protein bands that were specifically immunoprecipitated by the G1 mAb from DNT cell clone lysates, but not mutant clone lysates, were excised and the partial amino acid sequence was obtained using LC-mass spectrometry. Genome database analyses using the derived sequence information revealed microfibrillar-associated protein-4 (MFAP4) as the cell surface molecule on regulatory DNT cells specifically recognized by the G1 mAb. MFAP4 was validated as the DNT cell marker recognized by G1 mAb: (1) determination of the molecular size of the MFAP4 protein expressed by DNT cells; (2) specific recognition of rMFAP4 retrovirally expressed in CHO cell line by G1 mAb and conversely specific recognition of MFAP4 in G1⁺ DNT cell clones but not G1⁻ mutant DNT cell clones by anti-MFAP4 mAb; and (3) simultaneous recognition of the cell surface marker on the same activated DNT cell by G1 mAb and anti-MFAP4 Ab. The MFAP4 protein exhibits identical molecular size as the protein recognized by G1 mAb determined by western blot (FIG. 5); MFAP4 is specifically recognized by anti-MFAP4 mAb in G1⁺ DN T cell clones but not G1⁻ mutant clones; and anti-MFAP4 mAb and G1 mAb recognize a cell surface marker expressed on the surface of the same activated DNT cell (FIG. 6). The G1 mAb also recognizes a cell surface protein expressed on a subset of ex vivo-expanded human DNT cells (FIG. 4). Thus, MFAP4 is expressed on the cell surface of murine and human DNT cells, and G1 mAb immunostains this functional marker.

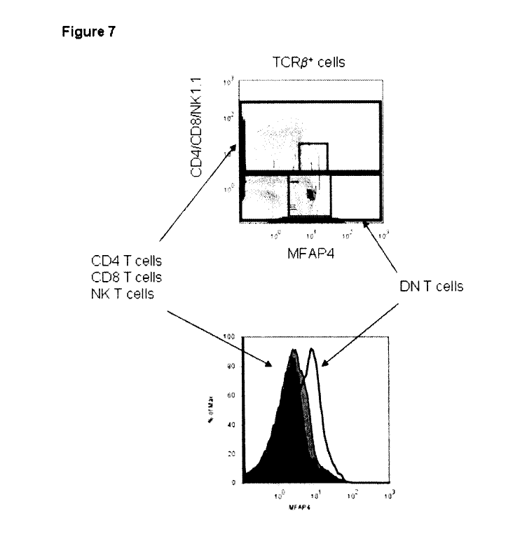

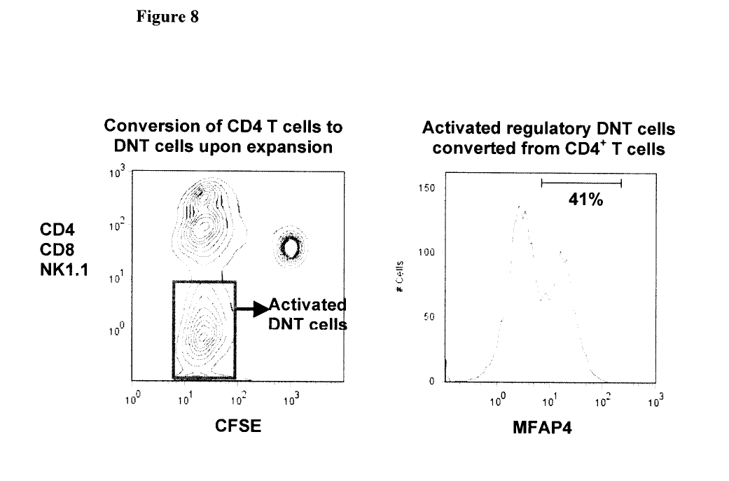

To determine whether MFAP4 is a specific functional marker on the cell surface of activated DNT cells, lymphocytes from B6.lpr mice were activated by alloantigen stimulation ([B6×Balb/c]_{F1} splenocytes) and cell surface expression of MFAP4 was determined on lymphocyte subsets by flow cytometry. MFAP4 is expressed on the surface of a subset of activated DNT cells (FIG. 7). Thus, G1 mAb can be used to mark and positively purify activated DNT cells. It was also found that a minor subset of CD4⁺, CD8⁺, and/or NK1.1⁺ T cells stained positive for MFAP4, suggesting that the G1 mAb could also be used as a novel functional marker for other regulatory T cell subsets. Another derivation of regulatory DNT cells is via conversion from CD4⁺ T cells that have undergone extensive cell proliferation stimulated by alloantigen stimulation ([B6×Balb/c]_{F1} mature bone marrow-derived dendritic cells) (10). It was found that a subset of DNT cells with suppressor function converted from CD4⁺ T cells expresses MFAP4 on their cell surface (FIG. 8).

MFAP4 is a 255-amino acid glycoprotein of the fibrinogen-related domain (FReD) family of proteins (11). It was first identified as a novel gene mapped to the chromosomal region associated with Smith-Magenis syndrome (SMS), a multiple congenital anomaly/mental retardation syndrome associated with deletion of chromosome 17p11.2 (12). MFAP4 contains a C-terminal fibrinogen-like domain (which has high similarity to the C-terminal halves of fibrinogen β and γ chains) and the N-terminal Arg-Gly-Asp (RGD) motif sequence that serves as an integrin-binding domain, suggesting that it is an extracellular matrix protein involved in cell adhesion or intercellular interactions. More recently, MFAP4 was shown to interact with pulmonary surfactant protein A (SP-A), a collectin involved in innate immunity against a broad range of micro-organisms by mediating microbial lysis and clearance, as well as modulating cytokine production and chemotaxis for phagocytes (13). Moreover, MFAP4 and SP-A co-localize both on the elastic fibres and lamina of pulmonary arteries of chronically inflamed lung tissue, consistent with a proposed role for MFAP4 in fixing collectins in the extracellular compartment during inflammation (13). Other members of the FReD family include fibrinogen-like protein 2/fibroleukin (FGL2), angiopoietins, ficolins, tachylectins and tenascins, and these molecules have been shown to play roles in innate and adaptive immune responses, including cell adhesion, cytokine production, and inflammation (13). With respect to an immunoregulatory role for proteins containing a fibrinogen-like domain, it has recently been shown that mice with targeted deletion of FGL2 spontaneously develop autoimmune glomerulonephritis, which correlated with increased T cell proliferation to alloantigens and increased numbers of Ab-producing B cells following immunization with T-independent Ags (14). Interestingly, FGL2 is produced by CD4⁺CD25⁺ regulatory T cells and their suppressive activity was significantly impaired by FGL2-deficiency (14). Moreover, anti-FGL2 Ab treatment completely inhibited Treg cell activity in vitro (14). Thus, MFAP4 as a FReD-containing protein may be important for DNT cell regulatory function.

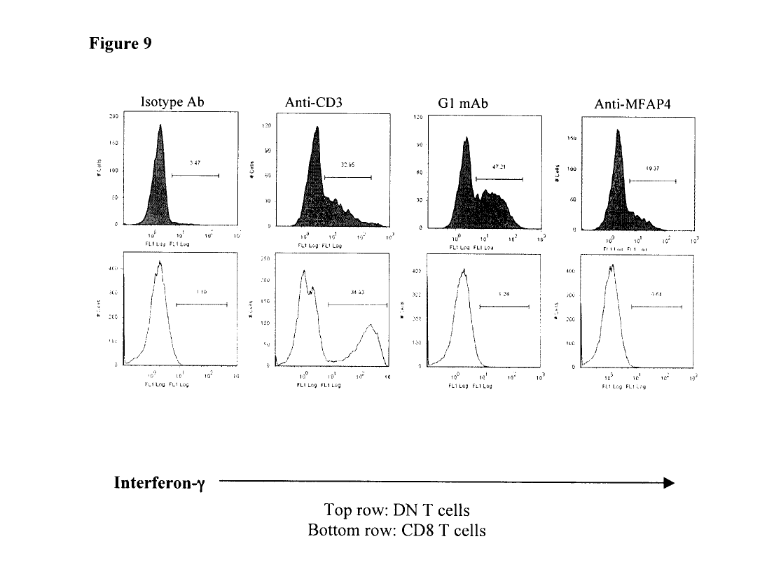

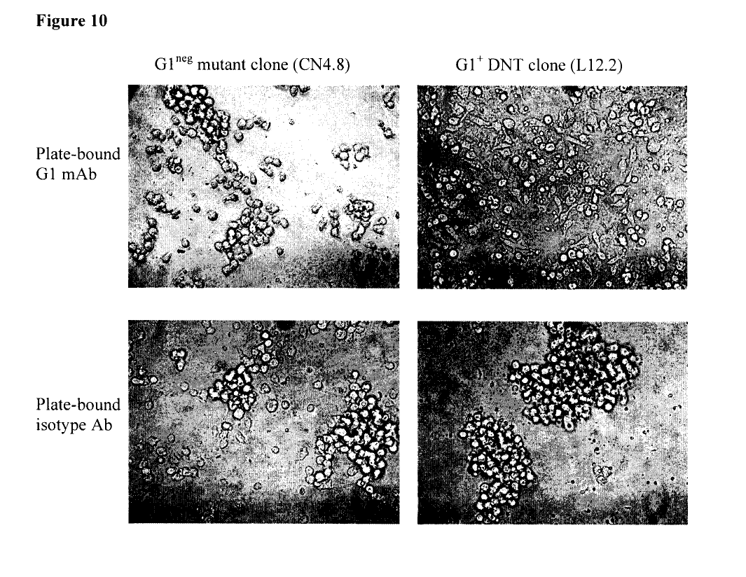

### 2. Treatment of DNT Cells with G1 mAb Induces Rapid Expression of Effector Cytokine IFNγ, Adherence and Morphological Activation (FIGS. 9-10)

It was next determined whether treatment of activated DNT cells with G1 mAb has important consequences for their function. G1 mAb-mediated crosslinking of MFAP4 on the cell surface of activated DN T cells by coating flat-bottom wells with G1 mAb induced rapid IFNγ expression (FIG. 9), an effector cytokine that has been reported to have a role in promoting tolerance in a murine allograft model. In addition, DNT cells treated with G1 mAb gain adherence and cell-spreading indicative of morphological/cytoskeletal activation (FIG. 10). Thus, G1 mAb can be used to induce rapid effector function by activated DNT cells.

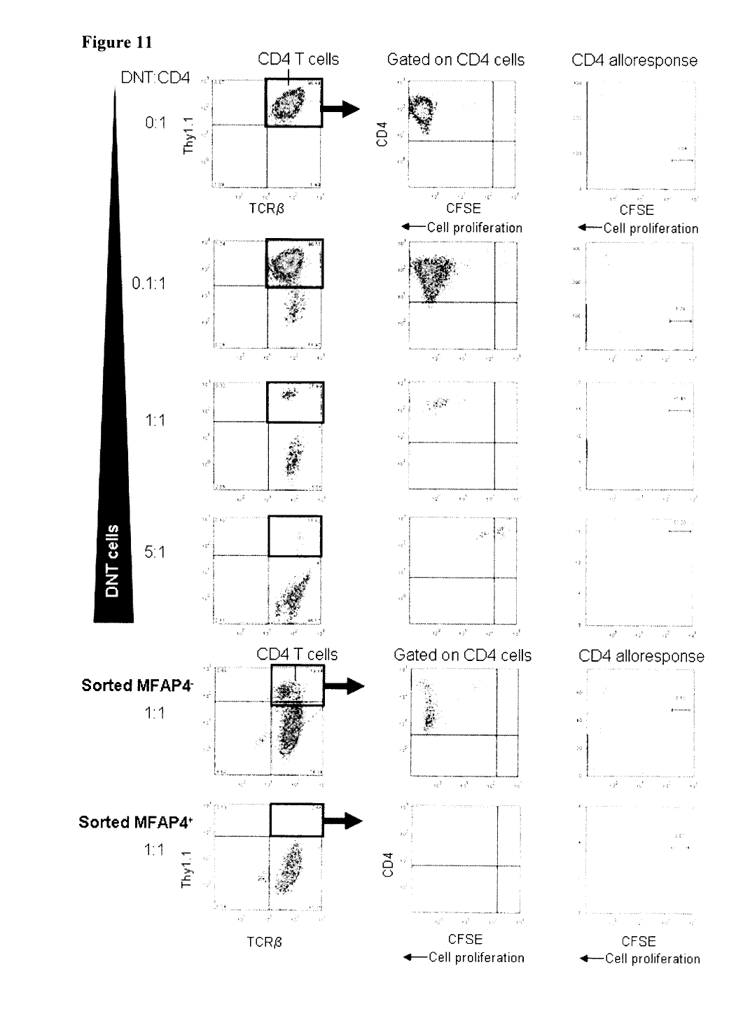

### 3. Enrichment of MFAP4-Positive DNT Cells Using G1 mAb Selects for Potent Suppressor Cells of Syngeneic Allo-Reactive T Cells (FIG. 11)

The utility of G1 mAb to select for regulatory DNT cells expressing the cell surface marker MFAP4 was determined by sorting MFAP4-negative and MFAP4-positive activated cells and examining their respective suppressor function against syngeneic allo-reactive CD4 T cells. Activated cells expressing MFAP4 were more potent at suppressing allo-reactive CD4 T cells in a mixed lymphocyte reaction compared with MFAP4-negative cells (FIG. 11): allo-reactive CD4 T cells were almost completely eliminated by MFAP4+ cells. Thus, MFAP4 is a cell surface marker recognized by G1 mAb and can be used to selectively enrich for potent DNT regulatory cells against allo-reactive T cells. As a minor subset of activated CD4+, CD8+, and/or NK1.1+ T cells have been found to express MFAP4 (FIG. 7), G1 mAb may also recognize this cell surface marker on other regulatory T cell subsets with potent suppressive efficacy.

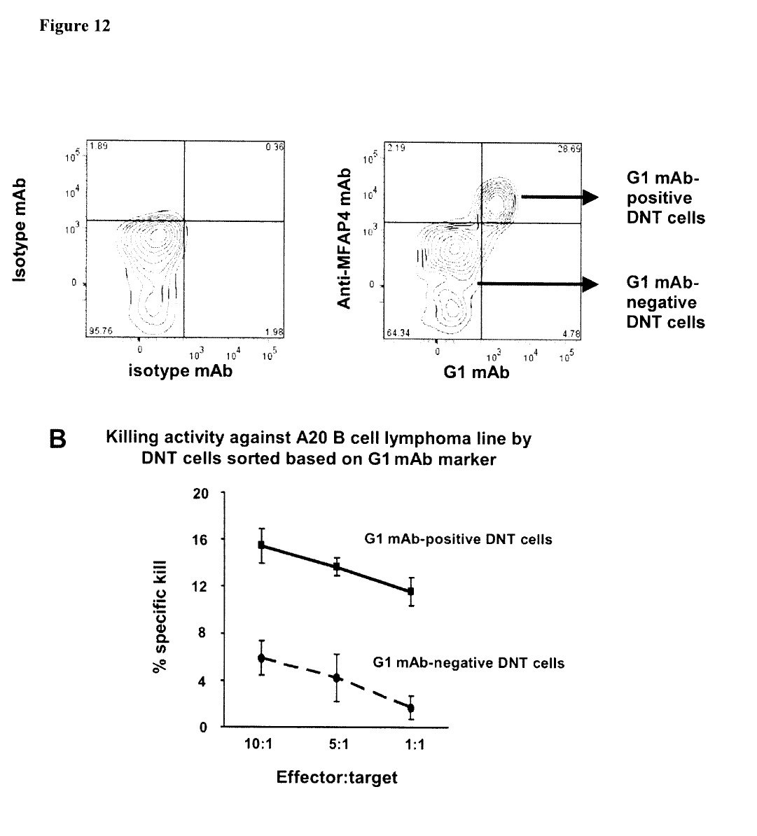

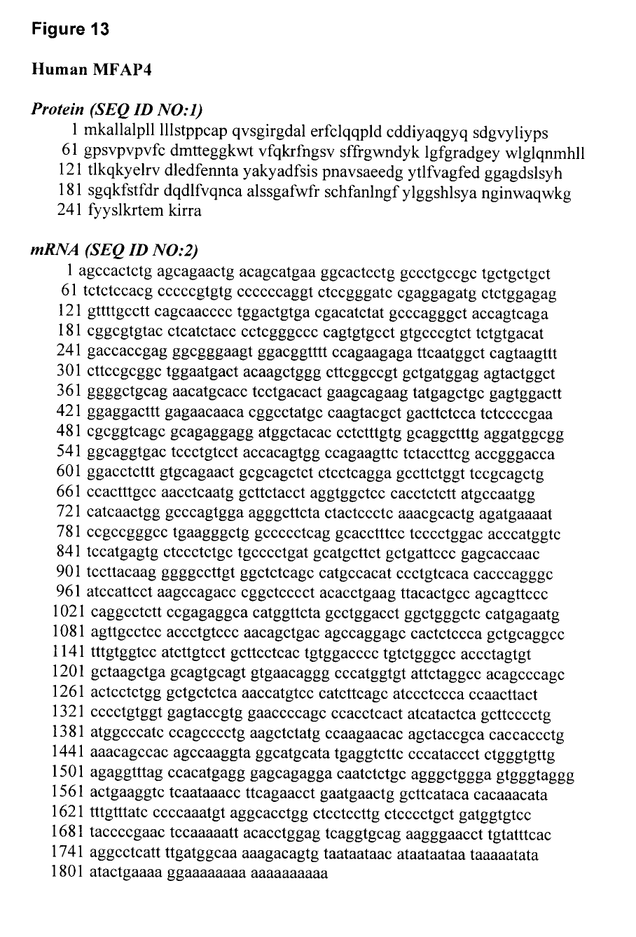

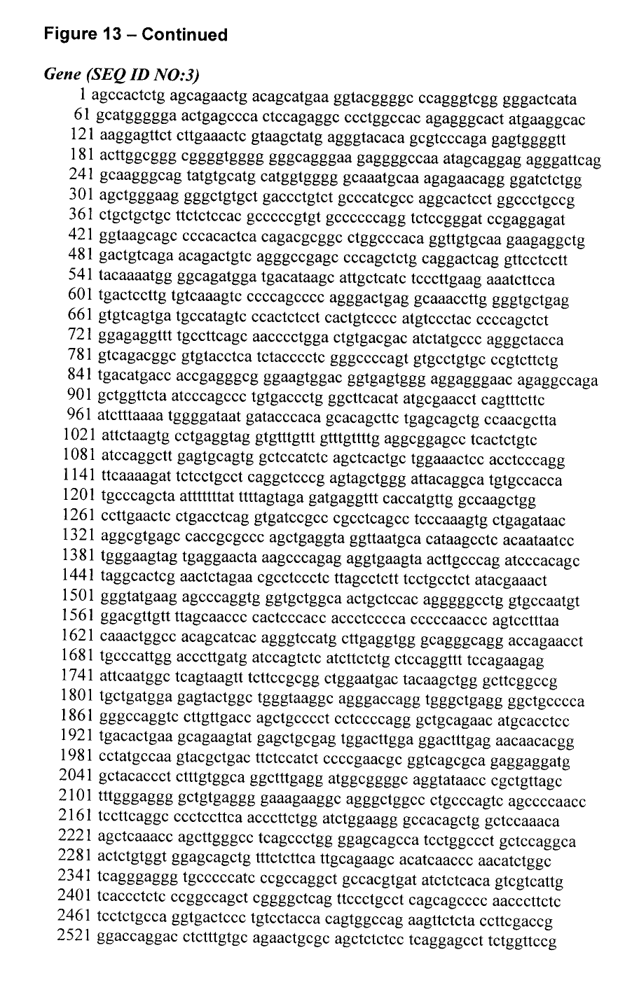

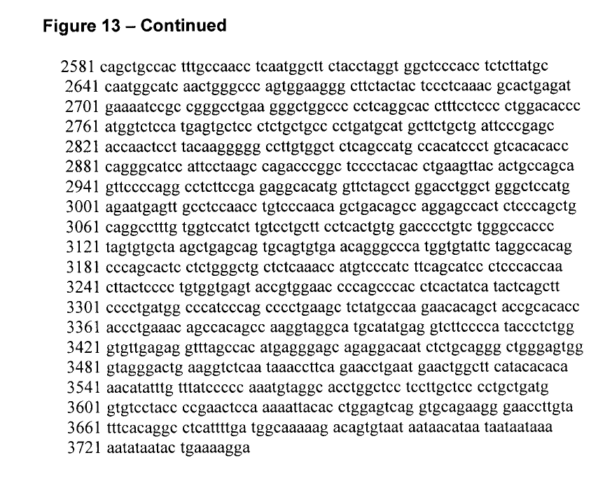

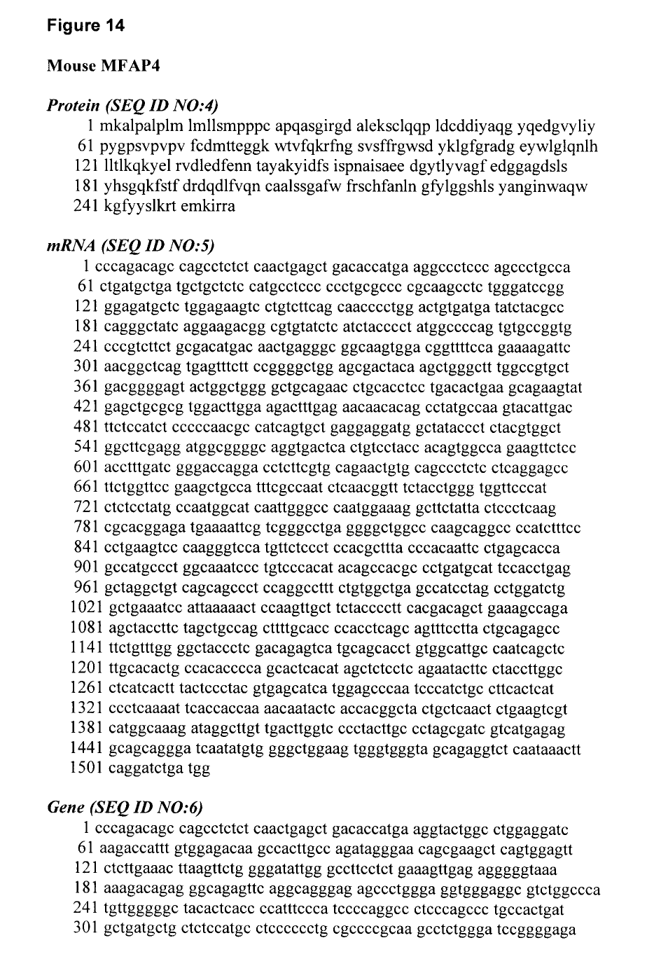

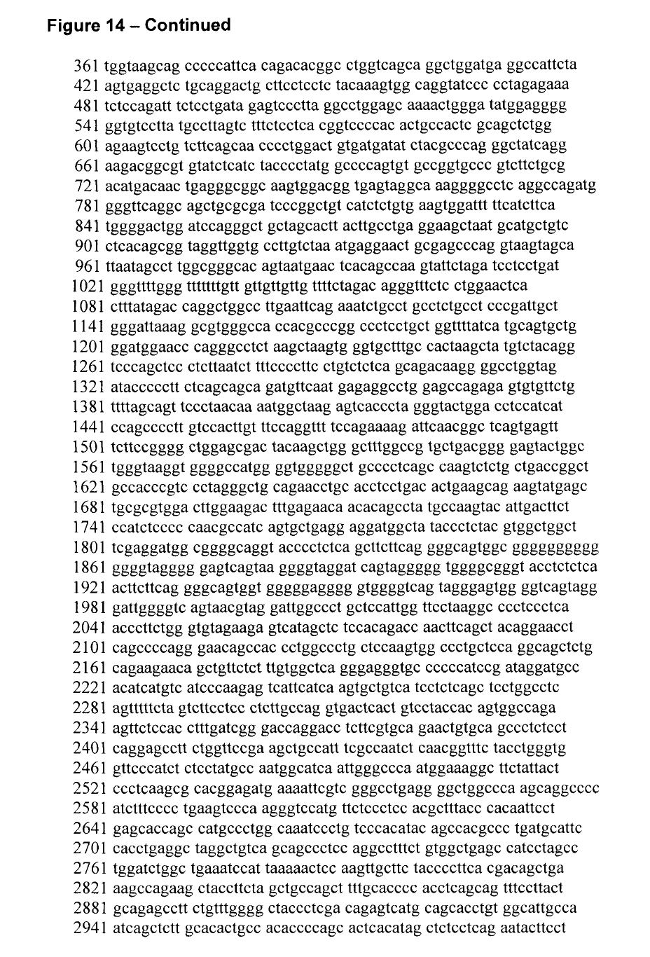

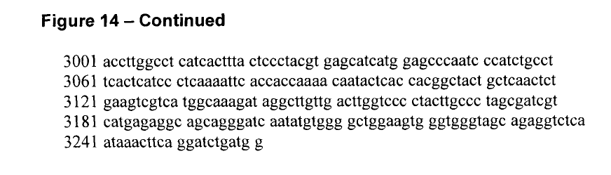

### 4. Enrichment of MFAP4-Positive DNT Cells Using G1 mAb Selects for Potent Anti-Tumor Cells (FIG. 12)

The utility of G1 mAb to select anti-tumor DNT cells expressing the cell surface marker MFAP4 was determined by sorting MFAP4-negative and MFAP4-positive DNT cells (FIG. 12A) and using them respectively as effectors against A20 B cell lymphoma cells. Activated cells expressing MFAP4 and selected based on positive binding with G1 mAb were more potent at killing tumor cells compared with MFAP4-negative, G1-negative cells (FIG. 12B). Thus, MFAP4 is a cell surface marker recognized by G1 mAb that can be used to selectively enrich for potent anti-tumor DNT cells.

### 5. Sequencing of Novel G1 Monoclonal Antibody

mRNA was extracted from hybridoma cell pellets. Total RNA was extracted from the pellets. cDNA was created from the RNA by reverse-transcription with an oligo(dT) primer. PCR reactions using variable domain primers were used to amplify both the VH and VL regions of the monoclonal antibody DNA. Positive VH and VL products were cloned into the Invitrogen sequencing vector pCR2.1 (Invitrogen: K2020-20) and transformed into TOP10 E. coli. Selected colonies were analyzed by colony PCR using vector specific primers M13 forward and M13 reverse. The colony PCR products were analysed as before by 1.5% agarose gel and ethidium bromide staining. Clones with positive bands around 500 bp were expanded to 5 ml culture in LB media overnight at 37° C. The plasmids were purified from the E. coli cultures using a plasmid miniprep kit (Qiagen) and the clones were sequenced.

CDRs were identified using the IMGT unique numbering system (Lefranc M.-P. et al. “IMGT unique numbering for immunoglobulin and Tcell receptor variable domains and Ig superfamily V-like domains” Dev. Comp. Immunol., 27, 55-77 (2003); Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008)).

The V_{H} nucleotide sequences for 6 clones are as shown in SEQ ID NO:7, 9, 11, 13, 15, and 17 and the V_{H} amino acid sequences for 6 clones are as shown in SEQ ID NO:8, 10, 12, 14 and 16 respectively.

Alignment of the V_{H} sequences is shown in FIG. 15. The consensus sequence is:

(SEQ ID NO: 19)
MDSRLNLVFLVLFIKGVQCEVKLVESGGGLVQPGRSLKLSCAASGFPF
SNYGMAWVRQAPTKGLAWVATISYDGRSTYYRDSVKGRFTISRNNAKS
TLYLQMDSLRSEDTATYYCVRHELPEDHWGQGVMVTVSSARTTAPPVY
PLAPGSL

The consensus heavy chain variable region comprises the amino acid sequence of SEQ ID NO:20. CDR1 of the V_{H} domain is as shown in SEQ ID NO:21; CDR2 of the V_{H} domain is as shown in SEQ ID NO:22; CDR3 of the V_{H} domain is as shown in SEQ ID NO:23.

The V_{L} nucleotide sequences for 9 clones are as shown in SEQ ID NOs: 24, 26, 28, 30, 32, 34, 36, 38 and 40 and the VL amino acid sequences are as shown in SEQ ID NOs:25, 27, 29, 31, 33, 35, 37, 39 and 41

Alignment of the V_{L} sequences is shown in FIG. 16. The consensus sequence is:

(SEQ ID NO: 42)
MVLISLLFWVYGTCGDIVMTQSPFSLAVSEGEMVTINCKSGQSLLSSG
NQKNYLAWYQQKPGQSPKLLIYYSSTRQSGVPDRFIGSGSGTDFTLTI
SDVQAEDLADYYCLQHYSSPFTFGSGTKLEIKRADAAPTVSIFPPSSK
LG

The consensus light chain variable region comprises amino acid sequence of SEQ ID NO:43. The CDR1 of V_{L} is shown in SEQ ID NO:44; the CDR2 of V_{L} is shown in SEQ ID NO:45; and the CDR3 of V_{L} is shown in SEQ ID NO:46.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Zhang Z X, Yang L, Young K J, DuTemple B, Zhang L. 2000. Identification of a previously unknown antigen-specific regulatory T cell and its mechanism of suppression. Nat. Med. 6:782-789.
2. Young K J, Yang L M, Phillips M J, Zhang L. 2002. Donor-lymphocyte infusion induces tolerance by activating systemic and graft-infiltrating double negative T regulatory cells. Blood 100:3408-3414.
3. Ford M S, Young K J, Zhang Z X, Ohashi P S, Zhang L. 2002. The immune regulatory function of lymphoproliferative double negative T cells in vitro and in vivo. J. Exp. Med. 196:261-267.
4. Young K J, DuTemple B, Phillips M J, Zhang L. 2003. Inhibition of graft-versus-host disease by double-negative regulatory T cells. J. Immunol. 171:134-141.
5. Chen W H, Ford M, Young K J, Cybulsky M, Zhang L. 2003. Role of double-negative regulatory T cells in long-term cardiac xenograft survival. J. Immunol. 170:1846-1853.
6. Ford M S, Chen W, Wong S, Li C, Vanama R, Elford A R, Asa S L, Ohashi P S, Zhang L. 2007. Peptide-activated double-negative T cells can prevent autoimmune type-1 diabetes development. Eur. J. Immunol. 37:2234-41.
7. Young K J, Kay L S, Phillips M J, Zhang L. 2003. Anti-tumor activity mediated by double-negative T cells. Cancer Res. 63:8014-8021.
8. Miyara M, and Sakaguchi S. 2007. Natural regulatory T cells: mechanisms of suppression. Trends Mol. Med. 13:108-16.
9. Chen W, Ford M S, Young K J, Zhang L. 2003. Infusion of in vitro-generated DN T regulatory cells induces permanent cardiac allograft survival in mice. Transplant Proc. 35:2479-80.
10. Zhang D, Yang W, Degauque N, Tian Y, Mikita A, and Zheng X-X. 2007. New differentiation pathway for double-negative regulatory T cells that regulates the magnitude of immune responses. Blood 109:4071.
11. Lausen M, Lynch N, Schlosser A, Tornøe I, Gjørup-Sækmose S, Teisner B, Willis A C, Crouch E, Schwaeble W, and Holmskov U. 1999. Microfibril-associated Protein 4 Is Present in Lung Washings and Binds to the Collagen Region of Lung Surfactant Protein D. J. Biol. Chem. 274:32234-32240.
12. Zhao Z, Lee C C, Jiralerspong S, Juyal R C, Lu F, Baldini A, Greenberg F, Caskey C T, Patel P I. 1995. The gene for a human microfibril-associated glycoprotein is commonly deleted in Smith-Magenis syndrome patients. Hum. Mol. Genet. 4:589-97.
13. Schlosser A, Thomsen T, Shipley J M, Hein P W, Brasch F, Tornøe I, Nielsen O, Skjødt K, Palaniyar N, Steinhilber W, McCormack F X, Holmskov U. 2006. Microfibril-associated protein 4 binds to surfactant protein A (SP-A) and colocalizes with SP-A in the extracellular matrix of the lung. Scand J Immunol. 64:104-16.
14. Shalev I, Liu H, Koscik C, Bartczak A, Javadi M, Wong K M, Maknojia A, He W, Liu M F, Diao J, Winter E, Manuel J, McCarthy D, Cattral M, Gommerman J, Clark D A, Phillips M J, Gorczynski R R, Zhang L, Downey G, Grant D, Cybulsky M I, Levy G. 2008. Targeted Deletion of fgl2 Leads to Impaired Regulatory T Cell Activity and Development of Autoimmune Glomerulonephritis. J. Immunol. 180:249-60

1. A method of detecting regulatory and/or anti-cancer cells comprising the steps of:
(1) contacting a test sample with a binding agent that binds specifically to MFAP4 on the cell to produce a binding agent-MFAP4 complex;
(2) detecting the amount of binding agent-MFAP4 complex in the test sample; and
(3) comparing the amount of binding agent-MFAP4 complex in the test sample to a control.

2. A method of selecting or enriching regulatory and/or anti-cancer cells comprising the steps:
(1) contacting a test sample with a binding agent that binds specifically to MFAP4 on the cell to produce a binding agent-MFAP4 complex; and
(2) selecting or enriching regulatory and/or anti-cancer cells by selecting the binding agent-MFAP4 complex from the test sample.

3. A method of activating regulatory and/or anti-cancer cells comprising:
contacting regulatory and/or anti-cancer cells with a binding agent that binds specifically to MFAP4 on the cell to crosslink MFAP4.

4. (canceled)

5. The method of claim 2, further comprising the step of activating the selected or enriched regulatory and/or anti-cancer cells by allowing a binding agent that binds specifically to MFAP4 on the cell to crosslink MFAP4.

6. (canceled)

7. The method of claim 2, wherein the regulatory T cells and/or anti-cancer cells comprise double negative T (DNT) cells.

8. The method of claim 7, wherein the double negative T cells comprise cells that are CD3⁺TCR⁺CD4⁻CD8⁻.

9. The method of claim 8, wherein the double negative T cells comprise cells that are CD3⁺αβTCR⁺CD4⁻CD8⁻.

10-14. (canceled)

15. The method of claim 2, wherein the binding agent comprises the light chain complementarity determining regions having the amino acid sequence of SEQ ID NOS: 44, 45, and/or 46, and/or the heavy chain complementarity determining regions having the amino acid sequence of SEQ ID NOS: 21, 22 and/or 23.

16-17. (canceled)

18. The method of claim 2, wherein the binding agent comprises a light chain variable region comprising the amino acid sequence of SEQ ID NO: 20 and a heavy chain variable region comprising the amino acid sequence of SEQ ID NO:43.

19. The method of claim 2, wherein the binding agent is an antibody.

20. The method of claim 19, wherein the antibody is a monoclonal antibody.

21. The method of claim 19, wherein the antibody is a humanized monoclonal antibody.

22. A method of treating or preventing an immune related disease comprising administering an effective amount of the regulatory cells selected by the method of claim 2.

23. The method of claim 22, wherein the immune related disease is autoimmune disease, allergy, graft rejection, graft versus host disease or infectious disease.

24-37. (canceled)

38. A binding agent comprising the light chain complementarity determining regions comprising the amino acid sequences defined by SEQ ID NOS: 44, 45 and/or 46 and/or the heavy chain complementarity determining regions comprising the amino acid sequence defined by SEQ ID NOS: 21, 22 and/or 23, or a variant thereof.

39. The binding agent according to claim 38 comprising the light chain variable region comprising the amino acid sequence of SEQ ID NO:43 and/or the heavy chain variable region comprising the amino acid sequence of SEQ ID NO:20, or a variant thereof.

40. The binding agent of claim 38, wherein the binding agent binds a protein comprising the amino acid sequence of SEQ ID NO:1 and/or SEQ ID NO:4.

41. The binding agent of claim 38, wherein the binding agent is an antibody.

42. The binding agent of claim 41, wherein the antibody is a monoclonal antibody.

43. The binding agent of claim 41, wherein the antibody is a humanized monoclonal antibody.

44. A method of modulating an immune response comprising administering an effective amount of the binding agent of claim 38.

45. (canceled)


SEQUENCE LISTING (46 sequences)

SEQ ID NO: 1 - protein, 255 aa, Homo sapiens
  MISC_FEATURE: MFAP4 amino acid
MKALLALPLLLLLSTPPCAPQVSGIRGDALERFCLQQPLDCDDIYAQGYQSDGVYLIYPSGPSVPVPVFCDMTTEGGKWTVFQKRFNGSVSFFRGWNDYKLGFGRADGEYWLGLQNMHLLTLKQKYELRVDLEDFENNTAYAKYADFSISPNAVSAEEDGYTLFVAGFEDGGAGDSLSYHSGQKFSTFDRDQDLFVQNCAALSSGAFWFRSCHFANLNGFYLGGSHLSYANGINWAQWKGFYYSLKRTEMKIRRA

SEQ ID NO: 2 - DNA, 1830 bp, Homo sapiens
  misc_feature: MFAP4 mRNA
agccactctgagcagaactgacagcatgaaggcactcctggccctgccgctgctgctgcttctctccacgcccccgtgtgccccccaggtctccgggatccgaggagatgctctggagaggttttgccttcagcaacccctggactgtgacgacatctatgcccagggctaccagtcagacggcgtgtacctcatctacccctcgggccccagtgtgcctgtgcccgtcttctgtgacatgaccaccgagggcgggaagtggacggttttccagaagagattcaatggctcagtaagtttcttccgcggctggaatgactacaagctgggcttcggccgtgctgatggagagtactggctggggctgcagaacatgcacctcctgacactgaagcagaagtatgagctgcgagtggacttggaggactttgagaacaacacggcctatgccaagtacgctgacttctccatctccccgaacgcggtcagcgcagaggaggatggctacaccctctttgtggcaggctttgaggatggcggggcaggtgactccctgtcctaccacagtggccagaagttctctaccttcgaccgggaccaggacctctttgtgcagaactgcgcagctctctcctcaggagccttctggttccgcagctgccactttgccaacctcaatggcttctacctaggtggctcccacctctcttatgccaatggcatcaactgggcccagtggaagggcttctactactccctcaaacgcactgagatgaaaatccgccgggcctgaagggctggccccctcaggcacctttcctcccctggacacccatggtctccatgagtgctccctctgctgcccctgatgcatgcttctgctgattcccgagcaccaactccttacaagggggccttgtggctctcagccatgccacatccctgtcacacacccagggcatccattcctaagccagacccggctcccctacacctgaagttacactgccagcagttccccaggcctcttccgagaggcacatggttctagcctggacctggctgggctccatgagaatgagttgcctccaccctgtcccaacagctgacagccaggagccactctcccagctgcaggcctttgtggtccatcttgtcctgcttcctcactgtggacccctgtctgggccaccctagtgtgctaagctgagcagtgcagtgtgaacagggcccatggtgtattctaggccacagcccagcactcctctgggctgctctcaaaccatgtcccatcttcagcatccctcccaccaacttactcccctgtggtgagtaccgtggaaccccagcccacctcactatcatactcagcttcccctgatggcccatcccagcccctgaagctctatgccaagaacacagctaccgcacaccaccctgaaacagccacagccaaggtaggcatgcatatgaggtcttccccataccctctgggtgttgagaggtttagccacatgagggagcagaggacaatctctgcagggctgggagtgggtagggactgaaggtctcaataaaccttcagaacctgaatgaactggcttcatacacacaaacatatttgtttatcccccaaatgtaggcacctggctcctccttgctcccctgctgatggtgtcctaccccgaactccaaaaattacacctggagtcaggtgcagaagggaaccttgtatttcacaggcctcattttgatggcaaaaagacagtgtaataataacataataataataaaaatataatactgaaaaggaaaaaaaaaaaaaaaaaa

SEQ ID NO: 3 - DNA, 3739 bp, Homo sapiens
  misc_feature: MFAP4 gene
agccactctgagcagaactgacagcatgaaggtacggggcccagggtcgggggactcatagcatgggggaactgagcccactccagaggcccctggccacagagggcactatgaaggcacaaggagttctcttgaaactcgtaagctatgagggtacacagcgtcccagagagtggggttacttggcgggcggggtgggggggcagggaagaggggccaaatagcaggagagggattcaggcaagggcagtatgtgcatgcatggtgggggcaaatgcaaagagaacaggggatctctggagctgggaaggggctgtgctgaccctgtctgcccatcgccaggcactcctggccctgccgctgctgctgcttctctccacgcccccgtgtgccccccaggtctccgggatccgaggagatggtaagcagccccacactcacagacgcggcctggcccacaggttgtgcaagaagaggctggactgtcagaacagactgtcagggccgagccccagctctgcaggactcaggttcctcctttacaaaatggggcagatggatgacataagcattgctcatctcccttgaagaaatcttccatgactccttgtgtcaaagtcccccagccccagggactgaggcaaaccttggggtgctgaggtgtcagtgatgccatagtcccactctcctcactgtccccatgtccctacccccagctctggagaggttttgccttcagcaacccctggactgtgacgacatctatgcccagggctaccagtcagacggcgtgtacctcatctacccctcgggccccagtgtgcctgtgcccgtcttctgtgacatgaccaccgagggcgggaagtggacggtgagtgggaggagggaacagaggccagagctggttctaatcccagccctgtgaccctgggcttcacatatgcgaacctcagtttcttcatctttaaaatggggataatgatacccacagcacagcttctgagcagctgccaacgcttaattctaagtgcctgaggtaggtgtttgtttgtttgttttgaggcggagcctcactctgtcatccaggcttgagtgcagtggctccatctcagctcactgctggaaactccacctcccaggttcaaaagattctcctgcctcaggctcccgagtagctgggattacaggcatgtgccaccatgcccagctaatttttttatttttagtagagatgaggtttcaccatgttggccaagctggccttgaactcctgacctcaggtgatccgcccgcctcagcctcccaaagtgctgagataacaggcgtgagccaccgcgcccagctgaggtaggttaatgcacataagcctcacaataatcctgggaagtagtgaggaactaaagcccagagaggtgaagtaacttgcccagatcccacagctaggcactcgaactctagaacgcctccctcttagcctctttcctgcctctatacgaaactgggtatgaagagcccaggtgggtgctggcaactgctccacagggggcctggtgccaatgtggacgttgttttagcaaccccactcccaccaccctccccacccccaacccagtcctttaacaaactggccacagcatcacagggtccatgcttgaggtgggcagggcaggaccagaaccttgcccattggacccttgatgatccagtctcatcttctctgctccaggttttccagaagagattcaatggctcagtaagtttcttccgcggctggaatgactacaagctgggcttcggccgtgctgatggagagtactggctgggtaaggcagggaccaggtgggctgaggggctgccccagggccaggtccttgttgaccagctgcccctcctccccagggctgcagaacatgcacctcctgacactgaagcagaagtatgagctgcgagtggacttggaggactttgagaacaacacggcctatgccaagtacgctgacttctccatctccccgaacgcggtcagcgcagaggaggatggctacaccctctttgtggcaggctttgaggatggcggggcaggtataacccgctgttagctttgggaggggctgtgaggggaaagaaggcagggctggccctgcccagtcagccccaacctccttcaggcccctccttcaacccttctggatctggaagggccacagctggctccaaacaagctcaaaccagcttgggcctcagccctggggagcagccatcctggccctgctccaggcaactctgtggtggagcagctgtttctcttcattgcagaagcacatcaacccaacatctggctcagggagggtgcccccatcccgccaggctgccacgtgatatctctcacagtcgtcattgtcaccctctcccggccagctcggggctcagttccctgcctcagcagccccaacccttctctcctctgccaggtgactccctgtcctaccacagtggccagaagttctctaccttcgaccgggaccaggacctctttgtgcagaactgcgcagctctctcctcaggagccttctggttccgcagctgccactttgccaacctcaatggcttctacctaggtggctcccacctctcttatgccaatggcatcaactgggcccagtggaagggcttctactactccctcaaacgcactgagatgaaaatccgccgggcctgaagggctggccccctcaggcacctttcctcccctggacacccatggtctccatgagtgctccctctgctgcccctgatgcatgcttctgctgattcccgagcaccaactccttacaagggggccttgtggctctcagccatgccacatccctgtcacacacccagggcatccattcctaagccagacccggctcccctacacctgaagttacactgccagcagttccccaggcctcttccgagaggcacatggttctagcctggacctggctgggctccatgagaatgagttgcctccaacctgtcccaacagctgacagccaggagccactctcccagctgcaggcctttgtggtccatcttgtcctgcttcctcactgtggacccctgtctgggccaccctagtgtgctaagctgagcagtgcagtgtgaacagggcccatggtgtattctaggccacagcccagcactcctctgggctgctctcaaaccatgtcccatcttcagcatccctcccaccaacttactcccctgtggtgagtaccgtggaaccccagcccacctcactatcatactcagcttcccctgatggcccatcccagcccctgaagctctatgccaagaacacagctaccgcacaccaccctgaaacagccacagccaaggtaggcatgcatatgaggtcttccccataccctctgggtgttgagaggtttagccacatgagggagcagaggacaatctctgcagggctgggagtgggtagggactgaaggtctcaataaaccttcagaacctgaatgaactggcttcatacacacaaacatatttgtttatcccccaaatgtaggcacctggctcctccttgctcccctgctgatggtgtcctaccccgaactccaaaaattacacctggagtcaggtgcagaagggaaccttgtatttcacaggcctcattttgatggcaaaaagacagtgtaataataacataataataataaaaatataatactgaaaagga

SEQ ID NO: 4 - protein, 257 aa, Mus musculus
  MISC_FEATURE: MFAP4 amino acid
MKALPALPLMLMLLSMPPPCAPQASGIRGDALEKSCLQQPLDCDDIYAQGYQEDGVYLIYPYGPSVPVPVFCDMTTEGGKWTVFQKRFNGSVSFFRGWSDYKLGFGRADGEYWLGLQNLHLLTLKQKYELRVDLEDFENNTAYAKYIDFSISPNAISAEEDGYTLYVAGFEDGGAGDSLSYHSGQKFSTFDRDQDLFVQNCAALSSGAFWFRSCHFANLNGFYLGGSHLSYANGINWAQWKGFYYSLKRTEMKIRRA

SEQ ID NO: 5 - DNA, 1513 bp, Mus musculus
  misc_feature: MFAP4 mRNA
cccagacagccagcctctctcaactgagctgacaccatgaaggccctcccagccctgccactgatgctgatgctgctctccatgcctcccccctgcgccccgcaagcctctgggatccggggagatgctctggagaagtcctgtcttcagcaacccctggactgtgatgatatctacgcccagggctatcaggaagacggcgtgtatctcatctacccctatggccccagtgtgccggtgcccgtcttctgcgacatgacaactgagggcggcaagtggacggttttccagaaaagattcaacggctcagtgagtttcttccggggctggagcgactacaagctgggctttggccgtgctgacggggagtactggctggggctgcagaacctgcacctcctgacactgaagcagaagtatgagctgcgcgtggacttggaagactttgagaacaacacagcctatgccaagtacattgacttctccatctcccccaacgccatcagtgctgaggaggatggctataccctctacgtggctggcttcgaggatggcggggcaggtgactcactgtcctaccacagtggccagaagttctccacctttgatcgggaccaggacctcttcgtgcagaactgtgcagccctctcctcaggagccttctggttccgaagctgccatttcgccaatctcaacggtttctacctgggtggttcccatctctcctatgccaatggcatcaattgggcccaatggaaaggcttctattactccctcaagcgcacggagatgaaaattcgtcgggcctgaggggctggcccaagcaggccccatctttcccctgaagtcccaagggtccatgttctccctccacgctttacccacaattcctgagcaccagccatgccctggcaaatccctgtcccacatacagccacgccctgatgcattccacctgaggctaggctgtcagcagccctccaggcctttctgtggctgagccatcctagcctggatctggctgaaatccattaaaaactccaagttgcttctaccccttcacgacagctgaaagccagaagctaccttctagctgccagcttttgcaccccacctcagcagtttccttactgcagagccttctgtttggggctaccctcgacagagtcatgcagcacctgtggcattgccaatcagctcttgcacactgccacaccccagcactcacatagctctcctcagaatacttcctaccttggcctcatcactttactccctacgtgagcatcatggagcccaatcccatctgccttcactcatccctcaaaattcaccaccaaaacaatactcaccacggctactgctcaactctgaagtcgtcatggcaaagataggcttgttgacttggtcccctacttgccctagcgatcgtcatgagaggcagcagggatcaatatgtggggctggaagtgggtgggtagcagaggtctcaataaacttcaggatctgatgg

SEQ ID NO: 6 - DNA, 3261 bp, Mus musculus
  misc_feature: MFAP4 gene
cccagacagccagcctctctcaactgagctgacaccatgaaggtactggcctggaggatcaagaccatttgtggagacaagccacttgccagatagggaacagcgaagctcagtggagttctcttgaaacttaagttctggggatattgggccttcctctgaaagttgagagggggtaaaaaagacagagggcagagttcaggcagggagagccctgggaggtgggaggcgtctggcccatgttgggggctacactcaccccatttcccatccccaggccctcccagccctgccactgatgctgatgctgctctccatgcctcccccctgcgccccgcaagcctctgggatccggggagatggtaagcagcccccattcacagacacggcctggtcagcaggctggatgaggccattctaagtgaggctctgcaggactgcttcctcctctacaaagtggcaggtatccccctagagaaatctccagatttctcctgatagagtcccttaggcctggagcaaaactgggatatggaggggggtgtccttatgccttagtctttctcctcacggtccccacactgccactcgcagctctggagaagtcctgtcttcagcaacccctggactgtgatgatatctacgcccagggctatcaggaagacggcgtgtatctcatctacccctatggccccagtgtgccggtgcccgtcttctgcgacatgacaactgagggcggcaagtggacggtgagtaggcaaaggggcctcaggccagatggggttcaggcagctgcgcgatcccggctgtcatctctgtgaagtggatttttcatcttcatggggactggatccagggctgctagcacttacttgcctgaggaagctaatgcatgctgtcctcacagcggtaggttggtgccttgtctaaatgaggaactgcgagcccaggtaagtagcattaatagccttggcgggcacagtaatgaactcacagccaagtattctagatcctcctgatgggttttgggtttttttgttgttgttgttgttttctagacagggtttctcctggaactcactttatagaccaggctggccttgaattcagaaatctgcctgcctctgcctcccgattgctgggattaaaggcgtgggccaccacgcccggccctcctgctggttttatcatgcagtgctgggatggaacccagggcctctaagctaagtgggtgctttgccactaagctatgtctacaggtcccagctccctcttaatcttttccccttcctgtctctcagcagacaaggggcctggtagatacccccttctcagcagcagatgttcaatgagaggcctggagccagagagtgtgttctgttttagcagttccctaacaaaatggctaagagtcaccctagggtactggacctccatcatccagccccttgtccacttgtttccaggttttccagaaaagattcaacggctcagtgagtttcttccggggctggagcgactacaagctgggctttggccgtgctgacggggagtactggctgggtaaggtggggccatggggtgggggctgcccctcagccaagtctctgctgaccggctgccacccgtccctagggctgcagaacctgcacctcctgacactgaagcagaagtatgagctgcgcgtggacttggaagactttgagaacaacacagcctatgccaagtacattgacttctccatctcccccaacgccatcagtgctgaggaggatggctataccctctacgtggctggcttcgaggatggcggggcaggtacccctctcagcttcttcaggggcagtggcggggggggggggggtagggggagtcagtaaggggtaggatcagtagggggtggggcgggtacctctctcaacttcttcaggggcagtggtgggggagggggtggggtcagtagggagtggggtcagtagggattggggtcagtaacgtaggattggccctgctccattggttcctaaggcccctccctcaacccttctgggtgtagaagagtcatagctctccacagaccaacttcagctacaggaacctcagccccagggaacagccaccctggccctgctccaagtggccctgctccaggcagctctgcagaagaacagctgttctctttgtggctcagggagggtgccccccatccgataggatgccacatcatgtcatcccaagagtcattcatcaagtgctgtcatcctctcagctcctggcctcagtttttctagtcttcctccctcttgccaggtgactcactgtcctaccacagtggccagaagttctccacctttgatcgggaccaggacctcttcgtgcagaactgtgcagccctctcctcaggagccttctggttccgaagctgccatttcgccaatctcaacggtttctacctgggtggttcccatctctcctatgccaatggcatcaattgggcccaatggaaaggcttctattactccctcaagcgcacggagatgaaaattcgtcgggcctgaggggctggcccaagcaggccccatctttcccctgaagtcccaagggtccatgttctccctccacgctttacccacaattcctgagcaccagccatgccctggcaaatccctgtcccacatacagccacgccctgatgcattccacctgaggctaggctgtcagcagccctccaggcctttctgtggctgagccatcctagcctggatctggctgaaatccattaaaaactccaagttgcttctaccccttcacgacagctgaaagccagaagctaccttctagctgccagcttttgcaccccacctcagcagtttccttactgcagagccttctgtttggggctaccctcgacagagtcatgcagcacctgtggcattgccaatcagctcttgcacactgccacaccccagcactcacatagctctcctcagaatacttcctaccttggcctcatcactttactccctacgtgagcatcatggagcccaatcccatctgccttcactcatccctcaaaattcaccaccaaaacaatactcaccacggctactgctcaactctgaagtcgtcatggcaaagataggcttgttgacttggtcccctacttgccctagcgatcgtcatgagaggcagcagggatcaatatgtggggctggaagtgggtgggtagcagaggtctcaataaacttcaggatctgatgg

SEQ ID NO: 7 - DNA, 455 bp, Rattus
  misc_feature: VH nucleic acid
atggactccaggctcaatttagttttccttgtccttttcataaaaggtgtccagtgtgaggtgaagctggtggagtctggtggaggcttagtgcagcctggaaggtccctgaaactctcctgtgcagcctcaggattccctttcagtaactatggcatggcctgggtccgccaggctccaacgaaggggctggcgtgggtcgcaaccattagttatgatggtcgtagcacttactatcgagactccgtgaagggccgattcactatctccagaaataatgcaaaaagcaccctatacctgcaaatggacagtctgaggtctgaggacacggccacttattactgtgtaagacatgagctgccggaggatcactggggccaaggagtcatggtcacagtctcctcagccagaacaacagccccacccgtctatccattggcccctggaagcttggg

SEQ ID NO: 8 - protein, 151 aa, Rattus
  MISC_FEATURE: VH amino acid
MDSRLNLVFLVLFIKGVQCEVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSSARTTAPPVYPLAPGSL

SEQ ID NO: 9 - DNA, 455 bp, Rattus
  misc_feature: VH nucleic acid
atggactccaggctcaatttagttttccttgtccttttcataaaaggtgtccagtgtgaggtgaagctggtggagtctggtggaggcttagtgcagcctggaaggtccctgaaactctcctgtgcagcctcaggattccctttcagtaactatggcatggcctgggtccgccaggctccaacgaaggggctggcgtgggtcgcaaccattagttatgatggtcgtagcacttactatcgagactccgtgaagggccgattcactatctccagaaataatgcaaaaagcaccctatacctgcaaatggacagtctgaggtctgaggacacggccacttattactgtgtaagacatgagctgccggaggatcactggggccaaggagtcatggtcacagtctcctcagccagaacaacagccccacccgtctatcccttggtccctggaagcttggg

SEQ ID NO: 10 - protein, 151 aa, Rattus
  MISC_FEATURE: VH amino acid
MDSRLNLVFLVLFIKGVQCEVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSSARTTAPPVYPLVPGSL

SEQ ID NO: 11 - DNA, 439 bp, Rattus
  misc_feature: VH nucleic acid
atggactccaggctcaatttagttttccttgtccttttcataaaaggtgtccagtgtgaggtgaagctggtggagtctggtggaggcttagtgcagcctggaaggtccctgaaactctcctgtgcagcctcaggattccctttcagtaactatggcatggcctgggtccgccaggctccaacgaaggggctggcgtgggtcgcaaccattagttatgatggtcgtagcacttactatcgagactccgtgaagggccgattcactatctccagaaataatgcaaaaagcaccctatacctgcaaatggacagtctgaggtctgaggacacggccacttattactgtgtaagacatgagctgccggaggatcactggggccaaggagtcatggtcacagtctcctcagccagaacaacagccccacccgtctatccactgg

SEQ ID NO: 12 - protein, 146 aa, Rattus
  MISC_FEATURE: VH amino acid
MDSRLNLVFLVLFIKGVQCEVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSSARTTAPPVYPL

SEQ ID NO: 13 - DNA, 456 bp, Rattus
  misc_feature: VH nucleic acid
atggactccaggctcaatttagttttccttgtccttttcataaaaggtgtccagtgtgaggtgaagctggtggagtctggtggaggcttagtgcagcctggaaggtccctgaaactctcctgtgcagcctcaggattccctttcagtaactatggcatggcctgggtccgccaggctccaacgaaggggctggcgtgggtcgcaaccattagttatgatggtcgtagcacttactatcgagactccgtgaagggccgattcactatctccagaaataatgcaaaaagcaccctatacctgcaaatggacagtctgaggtctgaggacacggccacttattactgtgtaagacatgagctgccggaggatcactggggccaaggagtcatggtcacagtctcctcagccagaacaacagccccacccgtctatcccctggtcccctggaagcttggg

SEQ ID NO: 14 - protein, 152 aa, Rattus
  MISC_FEATURE: VH amino acid
MDSRLNLVFLVLFIKGVQCEVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSSARTTAPPVYPLVPWKLG

SEQ ID NO: 15 - DNA, 456 bp, Rattus
  misc_feature: VH nucleic acid
atggactccaggctcaatttagttttccttgtccttttcataaaaggtgtccagtgtgaggtgaagctggtggagtctggtggaggcttagtgcagcctggaaggtccctgaaactctcctgtgcagcctcaggattccctttcagtaactatggcatggcctgggtccgccaggctccaacgaaggggctggcgtgggtcgcaaccattagttatgatggtcgtagcacttactatcgagactccgtgaagggccgattcactatctccagaaataatgcaaaaagcaccctatacctgcaaatggacagtctgaggtctgaggacacggccacttattactgtgtaagacatgagctgccggaggatcactggggccaaggagtcatggtcacagtctcctcagccagaacaacagccccacccgtctatccactggccccctggaagcttggg

SEQ ID NO: 16 - protein, 152 aa, Rattus
  MISC_FEATURE: VH amino acid
MDSRLNLVFLVLFIKGVQCEVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSSARTTAPPVYPLAPWKLG

SEQ ID NO: 17 - DNA, 455 bp, Rattus
  misc_feature: VH nucleic acid
atggagttcgggttcagcttggttttccttgtccttttcataaaaggtgtccagtgtgaggtgaagctggtggagtctggtggaggcttagtgcagcctggaaggtccctgaaactctcctgtgcagcctcaggattccctttcagtaactatggcatggcctgggtccgccaggctccaacgaaggggctggcgtgggtcgcaaccattagttatgatggtcgtagcacttactatcgagactccgtgaagggccgattcactatctccagaaataatgcaaaaagcaccctatacctgcaaatggacagtctgaggtctgaggacacggccacttattactgtgtaagacatgagctgccggaggatcactggggccaaggagtcatggtcacagtctcctcagccagaacaacagccccacccgtttatccactggcccctggaagcttggg

SEQ ID NO: 18 - protein, 151 aa, Rattus
  MISC_FEATURE: VH amino acid
MEFGFSLVFLVLFIKGVQCEVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSSARTTAPPVYPLAPGSL

SEQ ID NO: 19 - protein, 151 aa, Rattus; identical to SEQ ID NO: 8
  MISC_FEATURE: VH consensus

SEQ ID NO: 20 - protein, 116 aa, Rattus
  MISC_FEATURE: VH variable domain
EVKLVESGGGLVQPGRSLKLSCAASGFPFSNYGMAWVRQAPTKGLAWVATISYDGRSTYYRDSVKGRFTISRNNAKSTLYLQMDSLRSEDTATYYCVRHELPEDHWGQGVMVTVSS

SEQ ID NO: 21 - protein, 8 aa, Rattus
  MISC_FEATURE: VH CDR1
GFPFSNYG

SEQ ID NO: 22 - protein, 8 aa, Rattus
  MISC_FEATURE: VH CDR2
ISYDGRST

SEQ ID NO: 23 - protein, 9 aa, Rattus
  MISC_FEATURE: VH CDR3
VRHELPEDH

SEQ ID NO: 24 - DNA, 438 bp, Rattus
  misc_feature: VL nucleic acid
atggttctcatctccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 25 - protein, 146 aa, Rattus
  MISC_FEATURE: VL amino acid
MVLISLLFWVYGTCGDIVMTQSPFSLAVSEGEMVTINCKSGQSLLSSGNQKNYLAWYQQKPGQSPKLLIYYSSTRQSGVPDRFIGSGSGTDFTLTISDVQAEDLADYYCLQHYSSPFTFGSGTKLEIKRADAAPTVSIFPPSSKLG

SEQ ID NO: 26 - DNA, 438 bp, Rattus
  misc_feature: VL nucleic acid
atggttctcatgtccttgctgttctgggtatatggtacctgtggggacattgtgatgacccagtctccattctccctggctgtgtcagaaggagagatggtcactataaactgcaagtccggccagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 27 - protein, 146 aa, Rattus
  MISC_FEATURE: VL amino acid
MVLMSLLFWVYGTCGDIVMTQSPFSLAVSEGEMVTINCKSGQSLLSSGNQKNYLAWYQQKPGQSPKLLIYYSSTRQSGVPDRFIGSGSGTDFTLTISDVQAEDLADYYCLQHYSSPFTFGSGTKLEIKRADAAPTVSIFPPSSKLG

SEQ ID NO: 28 - DNA, 434 bp, Rattus
  misc_feature: VL nucleic acid
atggtccttatatccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagct

SEQ ID NO: 29 - protein, 144 aa, Rattus
  MISC_FEATURE: VL amino acid
MVLISLLFWVYGTCGDIVMTQSPFSLAVSEGEMVTINCKSGQSLLSSGNQKNYLAWYQQKPGQSPKLLIYYSSTRQSGVPDRFIGSGSGTDFTLTISDVQAEDLADYYCLQHYSSPFTFGSGTKLEIKRADAAPTVSIFPPSSK

SEQ ID NO: 30 - DNA, 437 bp, Rattus
  misc_feature: VL nucleic acid
atggttcttatgccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtgggtctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 31 - protein, 145 aa, Rattus
  MISC_FEATURE: VL amino acid
GSYALLFWVYGTCGDIVMTQSPFSLAVSEGEMVTINCKSGQSLLSSGNQKNYLAWYQQKPGQSPKLLIYYSSTRQSGVPDRFIGSGSGTDFTLTISDVQAEDLADYYCLQHYSSPFTFGSGTKLEIKRADAAPTVSIFPPSSKLG

SEQ ID NO: 32 - DNA, 438 bp, Rattus
  misc_feature: VL nucelic acid
atggtccttatgtccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 33 - protein, 146 aa, Rattus; identical to SEQ ID NO: 27
  MISC_FEATURE: VL amino acid

SEQ ID NO: 34 - DNA, 438 bp, Rattus
  misc_feature: VL  nucleic acid
atggttcttatgtccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 35 - protein, 146 aa, Rattus; identical to SEQ ID NO: 27
  MISC_FEATURE: VL amino acid

SEQ ID NO: 36 - DNA, 438 bp, Rattus
  misc_feature: VL nucleic acid
atggttcttatctccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 37 - protein, 146 aa, Rattus; identical to SEQ ID NO: 25
  MISC_FEATURE: VL amino acid

SEQ ID NO: 38 - DNA, 438 bp, Rattus
  misc_feature: VL nucleic acid
atggttctcatatccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 39 - protein, 146 aa, Rattus; identical to SEQ ID NO: 25
  MISC_FEATURE: VL amino acid

SEQ ID NO: 40 - DNA, 438 bp, Rattus
  misc_feature: VL nucleic acid
atggttcttatatccttgctgttctgggtatatggtacctgtggggacattgtgatgacccagtctccattctccctggctgtgtcagaaggagagatggtcactataaactgcaagtccggtcagagtcttttatccagtggaaaccaaaagaactacttggcctggtaccagcagaaaccagggcagtctcctaaactactgatctactattcatccactaggcaatcaggggtccctgatcgcttcataggcagtggatctgggacagacttcactctgaccatcagcgatgtgcaggctgaagacctggcagattattactgcctgcagcattacagctctccattcacgttcggctcagggacgaagttggaaataaaacgggctgatgctgcaccaactgtatccatcttcccaccatccagtaagcttggg

SEQ ID NO: 41 - protein, 146 aa, Rattus; identical to SEQ ID NO: 25
  MISC_FEATURE: VL amino acid

SEQ ID NO: 42 - protein, 146 aa, Rattus; identical to SEQ ID NO: 25
  MISC_FEATURE: VL consensus

SEQ ID NO: 43 - protein, 113 aa, Rattus
  MISC_FEATURE: VL variable region
DIVMTQSPFSLAVSEGEMVTINCKSGQSLLSSGNQKNYLAWYQQKPGQSPKLLIYYSSTRQSGVPDRFIGSGSGTDFTLTISDVQAEDLADYYCLQHYSSPFTFGSGTKLEIK

SEQ ID NO: 44 - protein, 12 aa, Rattus
  MISC_FEATURE: VL CDR1
QSLLSSGNQKNY

SEQ ID NO: 45 - protein, 3 aa, Rattus
  MISC_FEATURE: VL CDR2
YSS

SEQ ID NO: 46 - protein, 9 aa, Rattus
  MISC_FEATURE: VL CDR3
LQHYSSPFT